(19) United States
(12) Patent Application Publication
(10) Pub. No.: US 2021/0057072 A1
(43) Pub. Date: Feb. 25, 2021

(54) RESPONSIVE SERVER, SERVER SYSTEM AND SERVER METHOD FOR AUTOMATICALLY DISPENSING BASED ON COMPREHENSIVE MEDICATION AUTHORIZATION PROCESSING

(71) Applicant: PAN SOLUTIONS, LLC., Hanover Township, PA (US)

(72) Inventor: David Lee NOCKLEY, Plains, PA (US)

(73) Assignee: PAN SOLUTIONS, LLC., Hanover Township, PA (US)

(21) Appl. No.: 16/986,866

(22) Filed: Aug. 6, 2020

Related U.S. Application Data
(63) Continuation-in-part of application No. 15/674,515, filed on Aug. 10, 2017, now abandoned.
Application No. 15/674,515 is a continuation-in-part of application No. 13/524,543, filed on Jun. 15, 2012, now abandoned.
(60) Provisional application No. 61/457,839, filed on Jun. 16, 2011.

(51) Int. Cl. G16H 20/13; G16H 10/60; G07F 9/00; H04L 29/08; G06Q 40/08; A61J 1/03; A61J 7/00; B65D 83/04

(52) U.S. Cl. CPC G16H 20/13; G16H 10/60; G07F 9/009; G07F 9/002; B65D 83/04; G06Q 40/08; A61J 1/03; A61J 7/0076; H04L 67/125

(57) ABSTRACT

A responsive server, server system, and server method for automatically dispensing based on comprehensive medication authorization processing. The server may include performing automatic dispensing based on comprehensive medication information based on a complete medical history. In this respect, the disclosure provides prescription authorization checks that may include a medication search based on a complete medication history (i.e., from multiple sources, including those prescription claims not being processed through a prescription insurance carrier).

This is a Continuation-In-Part of U.S. application Ser. No. 15/674,515 filed Aug. 10, 2017, which is a Continuation-In-Part of U.S. application Ser. No. 13/524,543 filed Jun. 15, 2012, which claims the benefit of priority of Provisional U.S. Application No. 61/457,839 filed Jun. 16, 2011. The disclosures of the prior applications are hereby incorporated by reference herein in their entirety.

### BACKGROUND

### Technical Field

The present disclosure relates to a HIPAA-compliant (Federal Health Insurance Portability and Accountability Act of 1996) responsive server, server system and server method for comprehensive medication monitoring. In particular, the server, server system, and server method efficiently provide displayed information for determining whether a potential prescription should be authorized, while maintaining compliance with HIPAA, based on a more accurate determination of the medical history (including a more accurate medication history) of a person (patient) (by utilizing a complete medication history (i.e., from multiple sources, including those prescription claims not being processed through a prescription insurance carrier)).

### Related Art

Healthcare providers must not obtain or disclose unauthorized individually identifiable health information to comply with HIPAA. Noncompliance with HIPAA can result in criminal and civil penalties, even if the violation is unknowingly (and knowingly is defined as knowledge of the action—not knowledge of HIPAA). Thus, medication monitoring information must be securely protected, and, because of the HIPAA penalties, conventionally, medication monitoring information has not been shared.

In this respect, standalone HIPAA-compliant medication monitoring systems are generally known. For example, as shown in U.S. Patent Publication No. 2010/0299158 (Siegel), servers have been developed for using biometrics (e.g., fingerprinting, retinal scanning) for avoiding false identities in a standalone HIPAA-compliant medication monitoring system. See, e.g., paragraph [0007] of Siegel. In the standalone Siegel server, the prescription information (e.g., drug name and quantity) is electronically transmitted to the central server, along with the patient's BioID at the time of the prescription authorization check by the prescriber. See, e.g., paragraph [0036] of Siegel.

However, the conventional HIPAA-compliant servers (e.g., Siegel) fail to teach a comprehensive method that truly puts the “portability” into the HIPAA act. That is, Siegel does not provide a HIPAA-compliant server that can perform comprehensive prescription authorization checks based on portable HIPAA information from multiple sources (e.g., a local database, a comprehensive HIPAA-compliant server storing a database and, an insurance company database). That is, Siegel is only used by a single entity that can share information within the single entity. Siegel does not contemplate sharing information between multiple entities without violating HIPAA. Siegel does not provide a comprehensive check of other systems outside of its own (e.g., existing/legacy systems) so as to ensure accuracy in a practical sense.

In this respect, the conventional medication monitoring server technology (e.g., Siegel) does not provide comprehensive “portability” (of all medication history information) for the “portability” act (HIPAA). There is a need in the prior art for efficient, comprehensive HIPAA-compliant portability of medication information from a multitude of sources that prescriptions are authorized from (e.g., traditional retail chain pharmacies, independent pharmacies, and mail order pharmacies, etc.).

### Preventable Medication Errors (PME)

With an ever increasing number of prescriptions being filled (over 4 billion prescriptions being filled/dispensed in the United States annually), there is a corresponding increase in preventable medication authorization errors (PME) because of inaccurate/incomplete medication history information that cannot be shared. That is, with this increased market demand for prescription fills, patients have more options than ever to obtain medications (market supply increase). To illustrate this point, as an example, people (e.g., patients) often use multiple physicians to obtain prescriptions as well as may utilize multiple pharmacies to have the prescriptions filled. Unless the person uses the same pharmacy for every prescription they have filled, stays within the same retail chain, or uses the same insurance card for every prescription filled, there is currently no way for the conventional medication monitoring system servers to provide accurate (complete) medication monitoring result information to prescribers/pharmacists (by utilizing a complete medication history (i.e., from multiple sources, including those prescription claims not being processed through a prescription insurance carrier)).

Conventional server systems do not know (or even contemplate trying to know) medications/allergy conditions of a patient that has used other medication monitoring systems (i.e., other pharmacists or other prescribers systems). Thus, the conventional systems rely on inaccurate information. They do not take into account every medication that has been prescribed for a specific patient by another prescriber and/or dispensed by another pharmacist, let alone, other possible drug-interaction relevant medical information known to outside-of-their-network prescribers/pharmacists.

Thus, when a prescriber writes a prescription, he or she must do so with the assumption that the patient has provided a complete medical history to them including a complete list of all medications the patient is currently taking as well as what prescriber(s) has issued the prescription(s). However, people lie and forget, thus they cannot be trusted to provide complete medication history.

This creates a problem with the conventional technological medication monitoring (prescription authorization) server approach. With the conventional server approach, when a prescription is presented to a pharmacist, the pharmacist may perform a local (in-house) computer or server (retail chain) check of local/retail prescription records to determine if the prescription should be dispensed, such as, via Siegel's system. The in-house computer/server checks, based only on the local computer or retail server's information, for whether the prescription should be authorized.

Unfortunately, the conventional server's ability (to provide displayed information for indicating whether a potential prescription fill) is limited by the lack of completeness/accuracy (comprehensiveness) of the prescription records that are available. Individual pharmacies may keep local (in-house) records. Retail pharmacies and, separately, prescription insurance companies, may keep server records for the individual chain or company, respectively. In this respect, the conventional systems do not utilize a complete medication history (i.e., from multiple sources, including those prescription claims not being processed through a prescription insurance carrier)).

However, with the stiff HIPAA penalties acknowledged, none of these systems share; none has a truly-“portable” system allowing for portability/sharing of medication information, while maintaining HIPAA compliance. As a result, pharmacists must determine whether to fill a prescription based on incomplete/inaccurate information. The inaccurate/incomplete medication history profile results in pharmacists/prescribers, even using the conventional HIPAA-compliant Siegel server, unwittingly authorizing improper prescriptions based on inaccurate/incomplete medical history information. Ultimately, this inaccurate/incomplete medication history information results in the conventional servers providing information that leads to preventable medication errors (PME) (i.e., improperly authorized medications), which subsequently results in possible life-altering adverse drug events, and abuse/addiction, which results in an inefficient healthcare system (and corresponding increased healthcare costs).

### Adverse Drug Events (ADE) Caused by PME

ADE is defined in the art as: “an appreciably harmful or unpleasant reaction, resulting from an intervention related to the use of a medicinal product, which predicts hazard from future administration and warrants prevention or specific treatment, or alteration of the dosage regimen, or withdrawal of the product.” See Edwards, “Adverse Drug Reactions: Definitions, Diagnosis, and Management,” 2000, pages 1255-1259. Over 770,000 people are injured or die each year in hospitals from adverse drug events (ADEs), which may cost up to $5.6 million each year per hospital depending on hospital size. See “Reducing and Preventing Adverse Drug Events To Decrease Hospital Costs,” Agency for Healthcare Research and Quality, Publication No. 01-0020, March 2001. This estimate does not include ADEs causing admissions, malpractice and litigation costs, or the personal injury costs of patients. See Id. National hospital expenses to treat patients who suffer ADEs during hospitalization are estimated at between $1.56 and $5.6 billion annually. See Id.

Moreover, “patients who experienced adverse drug events (ADEs) were hospitalized an average of 8 to 12 days longer than patients who did not suffer ADEs, and their hospitalization cost $16,000 to $24,000 more.” See Id. “Anywhere from 28% to 95% of ADEs can be prevented by reducing medication errors through more accurate computerized monitoring systems.” See Id. Harmonized computerized medication order entry (via the disclosed shared server system) has the potential to prevent dose, frequency, and route errors. Hospitals can save direct costs by using a harmonized (universally-sharing) computerized server system.

Furthermore, “patients who experience ADEs have longer, more expensive hospitalizations than patients who do not suffer ADEs.” See Id. “For example, at LDS Hospital in Salt Lake City, researchers found that patients who experienced ADEs were hospitalized an average of 1 to 5 days longer than patients who did not suffer ADEs, with additional costs of up to $9,000.” See Id.

In this respect, “medication errors may occur at any point in the medication administration process—during ordering, transcription (the process of manually transferring the physician order onto medication sheets), dispensing, and administering medications.” See Id. However, “the majority of errors occur during the ordering and administration stages.” See Id. Thus, there is a need in the prior art to prevent these ordering and administration errors.

### Drug Abuse

In addition, drug overdose deaths, such as, opioid-involved deaths continue to increase in the United States. See Wolters Kluwer, “Costs of U.S. Prescription Opioid Epidemic Estimated at $78.5 Billion,” Sep. 14, 2016. For example, prescription opioid overdose, abuse, and dependence carries high costs for American society, with an estimated total economic burden of $78.5 billion. See Id. “More than 40 Americans die each day from overdoses involving prescription opioids. Families and communities continue to be devastated by the epidemic of prescription opioid overdoses.” See Id. “In nonfatal cases, costs for lost productivity—including reduced productive hours and lost production for incarcerated individuals—were estimated at about $20 billion.” See Id. “Nearly two-thirds of the total economic burden was due to health care, substance abuse treatment, and lost productivity for nonfatal cases.” See Id. “Fatal overdoses—including costs related to healthcare and lost productivity—accounted for $21.5 billion.” See Id. “There were also $7.7 billion in criminal justice-related costs—nearly all borne directly by state and local governments.” See Id. “In addition, the authors note reduced tax revenues due to opioid-related productivity losses.” See Id.

The scope of the abuse problem, as it pertains to prescription-related problems as described in the preceding paragraphs continues to exact devastating and unnecessary economic and social costs on American Society. While, for example, increased drug addiction demands greater attention to rehabilitation and recovery, the problem of pharmaceutical mismanagement and imprecise administration (based on inaccurate/incomplete medication profile history) is at the heart of the opioid crisis, as well as the more unintentional, growing adverse drug reaction problem facing Americans today.

These problems (ADEs and drug abuse) may be evident in the conventional technology when, as an example, a redundant claim is received by an insurance providers' main system for the same medication, which has already been distributed, thereby generating preventable, unwanted inefficiencies/costs. One approach to solve these problems of inaccuracy of medication/health information is for a prescriber to perform additional testing (that would be unnecessary with the server, server system and method disclosed below). The additional testing is inefficient and includes unnecessary costs (healthcare provider's bill for time spent conducting the test, testing equipment).

However, there exists within the conventional systems, no ability to accurately provide the most important prescriber/pharmacist information: regarding whether or not to authorize a prescription fill because there exists no uniform server system for prescribers and pharmacists in every sector, including retail, hospital, mail order, and so on, to corroborate/share the medication information they needed to share. With the HIPAA backdrop, they can't email each other the relevant information; they can't use social media. HIPAA requires no sharing of individually identifiable health information.

Accordingly, there is a need in the conventional technology to provide an efficient HIPAA-compliant server, server system and method to display prescription authorization check information that reduces, the frequency of, if not eliminates, these preventable medication authorization errors, thereby decreasing the negative impact these errors have on both patients (adverse health effects, drug abuse/addiction) and healthcare providers (efficiencies/costs). Embodiments of the present disclosure overcome the disadvantages of conventional medication monitoring servers systems described above and provide several advantages as will be described below.

### SUMMARY

To solve the above-described problems, ideally, a server performing automatic dispensing based on comprehensive medication information must be based on a complete medical history. In this respect, the disclosure provides prescription authorization checks that may include a medication search based on a complete medication history (i.e., from multiple sources, including those prescription claims not being processed through a prescription insurance carrier). However, to avoid HIPAA noncompliance, only information relevant to the specific prescription authorization request (that is to be prescribed and/or dispensed) should be utilized by the disclosed system, while maintaining the confidentiality of a patient's complete medication record, thereby adhering to HIPAA rules and regulations. By providing access to the relevant information, the disclosed server system may generate a complete medical profile based on information shared from different server systems that could then be utilized by the prescriber and pharmacist to determine the appropriateness of prescribing and dispensing a medication(s). The availability of such a server system to prescribers and its implementation into all pharmacies would provide a universally-comprehensive information database server system for prescribers and pharmacists to utilize, allowing them to prescribe, fill and dispense prescription utilizing more accurate information (by utilizing a complete medication history (i.e., from multiple sources, including those prescription claims not being processed through a prescription insurance carrier)). Automatic dispensing may include transmission of a control instruction to a dispensing machine that automatically dispenses medication corresponding to a prescription, or, transmission of the authorized prescription information to a printer such that the printer prints a label for the prescription, which is then placed on a container (e.g., bottle, pill container) to be filled.

According to an exemplary embodiment of the present invention, a server system for automatically dispensing after positive results (“no problems”) of a check for problems with filling a new prescription drug request or a refill of a previously-filled prescription drug request is provided. The server system may comprise a remote client terminal and a server. The remote client terminal may include a display, a network communications interface (configured to communicate with a universal prescription database server over a computer network), a memory (configured to store prescription drug records in a local database, each prescription drug record being stored in association with at least one unique patient identifier), and a hardware processor (CPU). The hardware processor of the remote client terminal may be programmed to: receive, based on user input, a prescription authorization request (also referred to as a claim submission) including unique patient identification information (e.g., unique identifier(s) of a patient, such as a social security number), prescription information (at least a drug identifier, and a drug quantity), and claim type information (e.g., either unique identification information of an insurance company or None or Cash (which each correspond to an indication that the patient has not presented insurance claim information of an insurance company/association that is associated with a BIN. Upon a click of a “Fill” button, the server system seamlessly determines, based on a check of multiple databases, and, if no problems, automatically fills (e.g., prints a prescription label signifying the prescription is filled/dispensed or about to be, or controls a filling machine to fill). In this respect, the system checks, a local database stored in the memory of the user terminal, whether one or more drug-related filling problems exist with filling the received prescription authorization request.

The hardware processor of the client terminal may be further programmed to: if at least one drug-related filling problems exists with the check of the local database, output to the display visibly-perceptible alert notification information identifying at least one of the one or more problems determined to exist, and if no drug-related filling problems exist with the check of the local database, and the remote client terminal is associated with a retail chain database server: (A) transmit, to the retail chain database server, the prescription authorization request including the associated unique patient identifier and the prescription information, (B) receive, from the retail pharmacy chain database server, a retail chain indication of whether any drug-related filling problems exist with the prescription authorization request, and (C) if the received retail chain indication indicates that at least one drug-related filling problems exists, output to the display visibly-perceptible alert notification information identifying at least one of the one or more problems determined to exist.

Further, if no drug-related filling problems exist with the check of the local database and the retail indication indicates that no drug-related filling problems exist with the prescription authorization request or the remote client terminal is not associated with a retail chain database server, the client hardware processor may be configured to: (A) transmit, to the universal prescription database server, the prescription authorization request including the associated unique patient identifier, the drug identifier and either the unique identifier of the insurance company or the indication that the patient does not have insurance, (B) receive, from the universal prescription database server, a universal indication of whether any drug-related filling problems exist with the prescription authorization request, and (C) if the received universal indication indicates that at least one drug-related filling problems exists, output to the display the visibly-perceptible alert notification information identifying at least one of the one or more problems determined to exist.

If the received universal indication indicates that no drug-related problems exist, the client hardware processor may be further configured to: (A) if the received prescription authorization request includes the indication (e.g., the claim type information) that the patient does not have insurance (e.g., NONE or similarly CASH), output to the display confirmation information indicating that no drug-related problems exist with filling/dispensing the prescription authorization request, and, in a preferred embodiment, automatically fill the prescription, (B) if the received prescription authorization request includes the unique identifier of the insurance company: (i) transmit, to a prescription database server of an insurance company, based on the unique identifier of the insurance company, the prescription authorization request including the associated unique patient identifier and the drug identifier, (ii) receive, from the prescription database server of the insurance company, an insurance indication of whether one or more drug-related filling problems exist with the prescription authorization request, and (iii) if the received insurance indication indicates that at least one drug-related filling problems exists, output to the display the visibly-perceptible alert notification information identifying at least one of the one or more problems determined to exist, and (C) if the received insurance indication indicates that no drug-related filling problems exist with the prescription authorization request output to the display the confirmation information indicating that no drug-related problems exist with filling/dispensing the prescription authorization request, and, preferably, automatically fill the prescription. Automatically fill the prescription means, as previously discussed, controlling a machine to automatically fill the prescription or causing a printer to print a label, which is the final step before a prescription is manually filled and dispensed. The hardware processor may be further configured to: upon entry of user input indicating that the prescription drug request has been automatically filled and dispensed, transmit, to the universal prescription database server, an indication of the filling and dispensing of the prescription drug request and additional information including, for example, the fill date, and the pharmacist identifier and/or prescriber identifier.

The server system may also include a universally-utilizable prescription database server comprising: a memory that stores a universally-utilizable prescription database, a network communications interface (configured to communicate with the remote client terminal over the computer network), and a hardware processor. The hardware processor may be programmed to: receive the prescription authorization request, over the computer network via the network communications interface from the remote client terminal, the prescription authorization request including the associated unique patient identifier information, the drug identifier, and the claim type (e.g., either the unique BIN identifier of the insurance company or the indication that the patient has not presented insurance (sometimes referred to in the disclosure as the patient does not have insurance), compare the drug identifier in the received prescription authorization request with one or more drug identifiers in existing prescription records associated with the same unique patient identifier information stored in the universal prescription database, if, based on the results of the comparison with the universal database server, at least one drug-related filling problem exists, transmit information identifying at least one of the one or more drug-related filling problems determined to exist, and if, based on the results of the comparison with the universal prescription database, no drug-related filling problems exist with the received prescription authorization request, transmit information indicating that no drug-related problems exist with filling the prescription authorization request. In this respect, the claim information can be submitted as a part of the automatic prescription fill process. For example, when a pharmacy fills the prescription with the claim type information being a nationally-recognized insurance company having its own BIN, the BIN can be included in the patient profile. Thus, when sent thru a switch server, the BIN is used to route the prescription authorization request to the insurance company. Further, in an exemplary embodiment, the universal database server may have a BIN assigned to it. Thus, the universal database server can be seamlessly implemented into the switch server as a supplemental universal database to cover, for example, information from independent pharmacies that are not associated with a retail chain. In this respect, more accurate information may be utilized when automatically filling and dispensing of prescriptions. As shown in the FIGS. below, the BIN corresponding to the insurance company of the claim type may be entered as a secondary check, on top of the primary BIN associated with the universal database server. With this functionality, a user may be provided automatic filling of prescriptions based on more accurate checks of problems, which were not possible with the conventional technology.

Further, the hardware processor of the server may be further configured to: upon receipt, from the remote terminal, of the indication that the requested prescription has been filled and dispensed and the additional information, update the universal prescription database to indicate that the requested prescription was filled and dispensed with the associated additional information.

### BRIEF DESCRIPTION OF THE DRAWINGS

Exemplary embodiments will be described with reference to the following drawings.

FIG. 1A shows a conventional server system 100 for medication monitoring for prescription fill authorization checks.

FIG. 1B shows a flowchart illustrating the processing a conventional server system for medication monitoring using patient information without universally-accepted insurance card information.

FIG. 1C shows a flowchart illustrating the processing a conventional server system for medication monitoring using patient information including universally accepted insurance card information.

FIG. 2 shows a server system 200 for medication monitoring-based prescription fill authorization checks in accordance with an exemplary embodiment of the present disclosure.

FIG. 3 shows a server system 300 illustrating the flow of data from a user terminal 310 at a remote location (e.g., shown as a pharmacy, but could also be a hospital/doctor's office (prescriber)) through a series of secure communication interfaces to a server check database 312 back to the remote terminal 310.

FIG. 4 shows a server system 400 illustrating the flow of data from a user terminal 410 at a remote location through a series of secure communication interfaces to a check server 412 back to the remote terminal 410.

FIG. 5A shows the hardware of a user terminal 200, which may be located at a remote location.

FIG. 5B shows an authorization check server 520 and a profile database server 540 of the embodiments of anyone of FIGS. 2-4.

FIG. 5C shows a high-level flowchart showing processing executed by user terminal 200 according to an exemplary embodiment of the disclosure for performing processing of for a pharmacy location (e.g., a brick-and-mortar independent pharmacy).

FIG. 5D shows a screenshot of a display (output device 217) of the user terminal 200 showing a “highlighted” icon with an “arrow” over it, which is the icon to start the process.

FIG. 5E shows a screenshot of a display (output device 217) of the user terminal 200 displaying how to login, which is to authenticate the user via the login function 212a.

FIG. 5F shows a screenshot of a display (output device 217) of the user terminal 200 displaying where to enter login credentials.

FIG. 5G shows a screenshot of a display (output device 217) of the user terminal 200 displaying, once login has been authenticated, an Rx Processing Tasks Screen 104 including a search button.

FIG. 5H shows a screenshot of a display (output device 217) of the user terminal 200 displaying the patient's profile.

FIG. 5I shows a screenshot of a display (output device 217) of the user terminal 200 displaying the patient's insurance information that is on file (which is via the “insurance information” tab on the bottom left (highlighted)).

FIG. 5J shows a screenshot of a display (output device 217) of the user terminal 200 showing how a pharmacist enters a prescription for filling that is NOT using our system.

FIG. 5K shows a screenshot of a display (output device 217) of the user terminal 200 displaying, once login has been authenticated, an Rx Processing Tasks Screen 108 including a search button.

FIG. 5L shows a screenshot of a display (output device 217) of the user terminal 200 displaying, an Electronic Claims Log screen 109.

FIG. 5M is similar to FIG. 5H and shows a screenshot of a display (output device 217) of the user terminal 200 displaying the patient's profile.

FIG. 5N shows a screenshot of a display (output device 217) of the user terminal 200 showing how a pharmacist enters a prescription for filling using an embodiment of the disclosed server.

FIG. 5O shows a screenshot of a display (output device 217) of the user terminal 200 showing a rejection from the universally-utilizable server system.

FIG. 5P shows a respective screenshot of a display (output device 217) of the user terminal 200 showing a rejection from the universally-utilizable server system.

FIG. 5Q shows a screenshot of a display (output device 217) of the user terminal 200 showing the patient profile with the patient's social security number entered on their profile.

FIG. 5R shows a screenshot of a display (output device 217) of the user terminal 200 showing the Electronic Claims Log screen showing the claim for rx#6066197 processed through SCRIPTCHECK successfully.

FIG. 6A shows a high-level flowchart showing some of the processing executed by an embodiment of the discloser related to a terminal device of a prescriber utilizing a universal database server according to an exemplary embodiment.

FIG. 6B shows an exemplary embodiment of a universal prescription database including attributes or characteristics of medication data (including prescriptions and disbursements each including associated patient info) stored in the universal prescription database, and communication interfaces between a plurality of devices.

FIGS. 7A and 7B show a high-level flowchart showing processing executed by a script server utilizing a universal database server according to an exemplary embodiment.

FIG. 8A is a sample screenshot showing displayable information sent as a response returned to a remote terminal from the script server/universal prescription database server illustrating an “EARLY REFILL” problem warning and the selectable options available to the user terminal.

FIG. 8B show exemplary screenshots of information displayed in a remote terminal, in response to a user's selection of a “PAYMENT INFORMATION” option of FIG. 8A.

FIG. 8C is a sample screen shot showing confirmation of the data stored by the universal prescription database server for reporting purposes when a pharmacist chooses the “PHARMACIST OVERRIDE” option to override the early refill warning of FIG. 8A.

FIG. 9A is a sample screen shot showing displayed information sent as a response returned to a remote terminal from the script server/universal prescription database server illustrating a “DUPLICATE THERAPY” problem warning and the selectable options available to the user terminal.

FIG. 9B shows exemplary screenshots of information displayed in a remote terminal, in response to a user's selection of a “PAYMENT INFORMATION” option of FIG. 9A where the patient does not have a universally-accepted insurance card.

FIG. 9C is a sample screen shot showing confirmation of the data stored by the universal prescription database server for reporting purposes when a pharmacist chooses the “PHARMACIST OVERRIDE” option to override the “DUPLICATE THERAPY” warning of FIG. 9A.

FIG. 10A is a sample screen shot illustrating displayable information sent as a response returned to a remote terminal from the script server/universal prescription database server illustrating an “DRUG-DRUG INTERACTION” (INTX) problem warning and the selectable options available to the user terminal.

FIG. 10B shows an exemplary screenshot of information displayed in a remote terminal, in response to a user's selection of a “PAYMENT INFORMATION” option of FIG. 10A.

FIG. 10C is a sample screen shot showing confirmation of the data stored by the universal prescription database server for reporting purposes when a pharmacist chooses the “PHARMACIST OVERRIDE” option to override the “DRUG-DRUG INTERACTION” (INTX) warning of FIG. 10A.

FIG. 11A shows a sample screenshot illustrating information displayed by the remote terminal, which is received in a response returned to the remote terminal from the script server/universal prescription database, the displayed information showing a “DRUG-DISEASE STATE INTERACTION” (INTX) problem warning.

FIG. 11B is a sample screen shot showing confirmation of the data stored by the universal prescription database server for reporting purposes when a pharmacist chooses the “PHARMACIST OVERRIDE” option to override the “DRUG-DRUG DISEASE STATE INTERACTION” (INTX) warning of FIG. 11A.

FIG. 12A shows a sample screenshot illustrating information displayed by the remote terminal, which is received in a response returned to the remote terminal from the script server/universal prescription database, the displayed information showing a “DRUG ALLERGY” problem warning and the selectable options available to the user terminal.

FIG. 12B is a sample screenshot showing confirmation of the data stored by the universal prescription database server for reporting purposes when a pharmacist chooses the “DELETE ALLERGY” option to override the “DRUG ALLERGY” warning of FIG. 12A.

FIG. 12C is a sample screenshot showing confirmation of the data stored by the universal prescription database server for reporting purposes when a pharmacist chooses the “PHARMACIST OVERRIDE” option to override the “DRUG ALLERGY” warning of FIG. 12A.

FIG. 13 is a sample screenshot illustrating the response returned to a remote terminal from the universal prescription database server(s) indicating that a complete search was performed, how many errors were overridden, and what those error were.

### DETAILED DESCRIPTION OF EXEMPLARY EMBODIMENTS

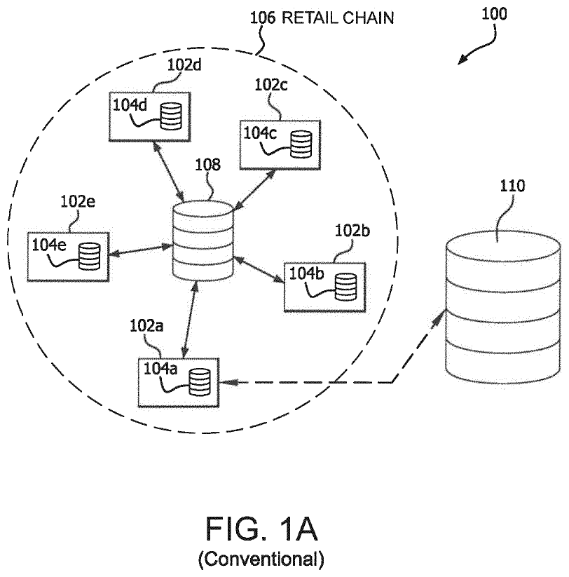

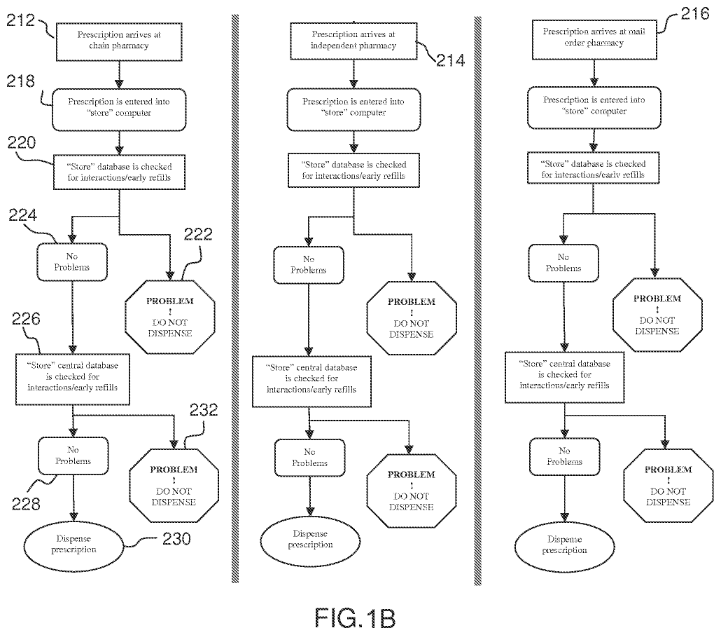

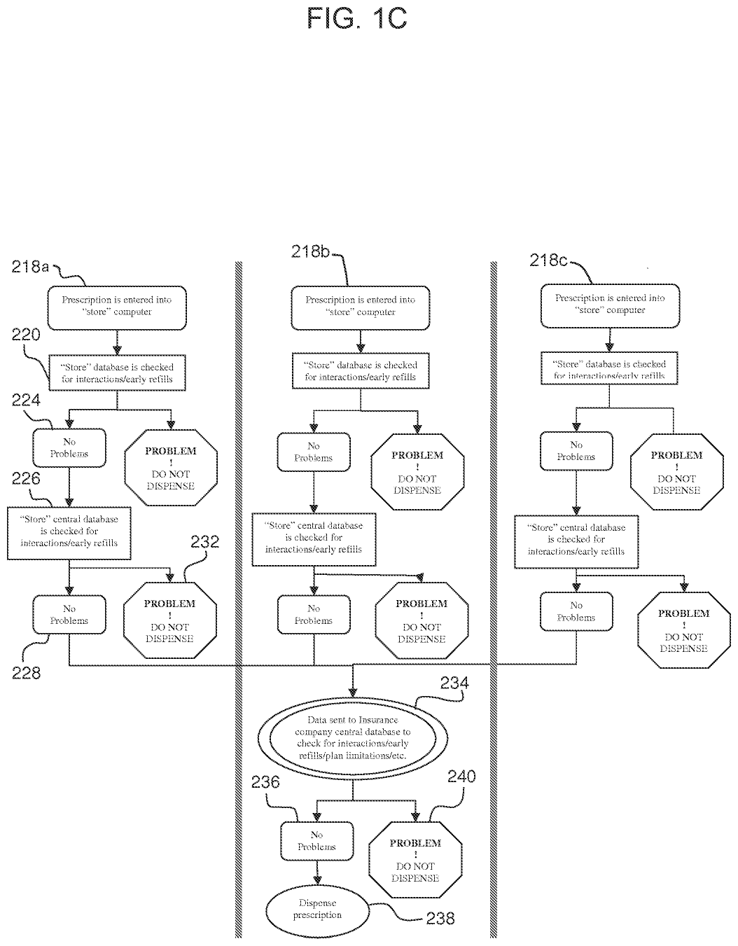

FIG. 1A shows a conventional server system 100 for medication monitoring for prescription fill authorization checks. In particular, an individual pharmacy terminal device 102a has a local database 104a for storing prescription records for customers. The local database 104a may be stored on a memory of the terminal device 102a. The memory device may be any type of conventional memory. Pharmacy terminal 102a is shown in FIG. 1A as a retail pharmacy that is part of a retail chain 106 of pharmacy terminals 102a-102e. However, the pharmacy terminal 102a could also be a similar system of an already-connected group of terminal devices associated with one of: a mail order pharmacy, a hospital pharmacy, an independent pharmacy, and so on with very minor modifications.

As shown in FIG. 1A, pharmacy terminal 102a is part of a retail chain 106 that includes other member pharmacy terminals 102b, 102c, 102d, 102e. Each of the other member pharmacy terminals 102b-102e may have their own respective local databases 104b, 104c, 104d, 104e for storing records of prescriptions filled at the respective member pharmacies. The retail chain 106 also includes a central database server 108 that is utilizable from each of the retail chain 106 member pharmacy terminals. The central database server 108 contains records of prescriptions filled by a particular customer at any of the retail chain member pharmacies 102a-102e. However, this is still incomplete, as it does not account for prescriptions filled by the customer at a different pharmacy that is not connected to the central database 108.

In the conventional example of FIG. 1A, if the customer filling a prescription at the pharmacy, via terminal 102a, is using a universal insurance card, than a third database 110 may be accessed (based on the universal insurance card information). The insurance company database 110 is routinely checked by computers, when a pharmacist enters universal insurance card information received by a customer, when submitting an insurance claim. The insurance company database 110 includes records of prescriptions filled anywhere, as long as an insurance claim was submitted in connection with the prescription.

Once a prescription is presented at pharmacy 102a, the local database 104a, the retail chain central database 108, and optionally the insurance company database 110 (if a universal insurance card was presented) are all updated to record a record of the prescription.

FIG. 1B shows a conventional method of displaying information indicating whether or not a prescription fill request is authorized by a user terminal, in the case that a universally-accepted prescription insurance card is not used. In a real world exemplary scenario, a patient visits a medical services provider that prescribes medicine (e.g., a doctor, a nurse practitioner), herein after a “prescriber.” The prescriber may ask the patient how the prescription information should be delivered to the pharmacy, and, which one. However, many times, the patient just receives a paper of the prescription information. Regardless of the method of transmittal from the prescriber to the pharmacist (e.g., patient hand-delivery, phone authorization, fax, electronic prescription sent), the prescription information is received/entered into a pharmacy terminal, which may be of several different types. If the pharmacy terminal is a part of a traditional retail pharmacy chain (e.g., terminal 102a in chain 106 of FIG. 1A), the method continues at step 112 along the left-most column of FIG. 1B. If the pharmacy is an independent pharmacy, the method continues at step 114 along the middle column of FIG. 1B. Finally, if the pharmacy is a mail order pharmacy, the method continues at step 116 along the right-most column of FIG. 1B.

At the retail chain pharmacy terminal device, the prescription is entered (Step 118), and checked against the local store database 104a (Step 120) to determine if there are any problems with filling the prescription (e.g., drug allergies, negative drug-disease state interactions, negative drug-drug interactions, duplicate therapies, early refills (abuse/overuse of a medication), and other potential negative problems). If there are any problems identified from the check/search of local database 104a, a warning alert is issued, in the form of displayed information, and the prescription is not filled (Step 122). If no problems are identified (Step 124), the retail chain central database 108 is checked (Step 126). If are still no problems identified in the records check of central database 108 (Step 126), the prescription is authorized to be filled, and dispensed (Steps 228 and 230). On the other hand, if the central database 108 records do identify a problem (Step 126), than an alert is issued and the prescription is not filled (Step 132).

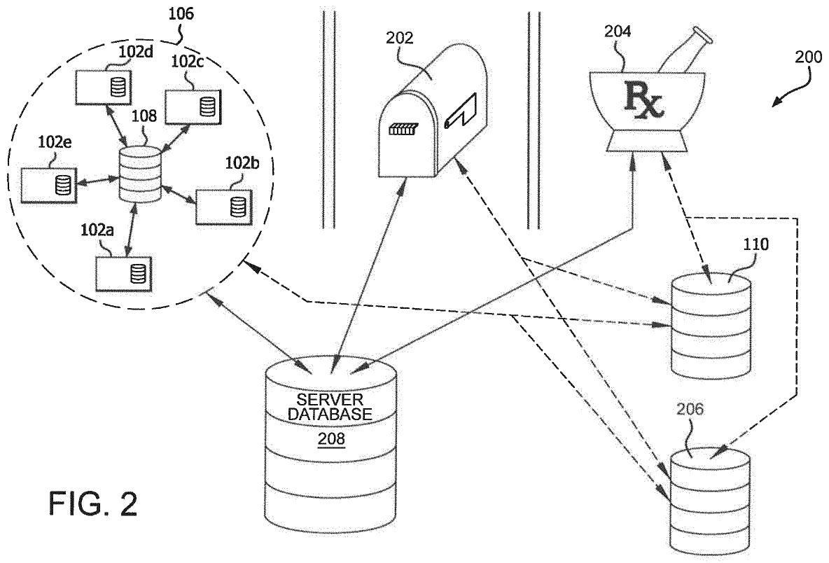

In an independent pharmacy or a mail-order pharmacy (as shown in the middle column and right-most column, respectively, of FIG. 1B), the method proceeds similarly with the exception that there may be no central database server to check. That is, the independent pharmacy may not be a part of a retail chain. The mail-order pharmacy could be a part of a retail chain, or could not. Since the method proceeds similarly for independent and mail order pharmacies, a description of the individual steps will not be repeated here. However, importantly, in the conventional system, the medication monitoring information is not shared between the three columns shown in FIG. 1B. That is, each of the individual systems (retail chain, independent, mail order) do not interact. Because of such, the medication monitoring information is inaccurate as it is not a complete representation of all possible prescriptions the patient may have received (had dispensed to him). That is, it does not utilize a complete medication history (i.e., from multiple sources, including those prescription claims not being processed through a prescription insurance carrier)). In other words, problems with drug allergies, negative drug-disease state interactions, negative drug-drug interactions, duplicate therapies, early refills (overuse of a medication), and other potential negative problems could occur and remain undetected if a patient merely uses a first pharmacy, such as a retail pharmacy (left-most column of FIG. 2) for one prescription, and a different pharmacy such as a mail order (right-most column) or independent pharmacy (middle column) for another prescription.

The situation described above is somewhat improved if a patient uses a universal insurance card, although important disadvantages remain, as will be described in connection with FIG. 1C. FIG. 1C shows a flowchart illustrating the processing of a conventional server system for medication monitoring using patient information including universally accepted insurance card information. The first portion of the processing, from a prescription being entered/received by a remote pharmacy terminal through the pharmacy central database being checked (Steps 120, 126, 128, 132) are substantially the same as described in connection with FIG. 1B, and so a detailed description thereof need not be repeated here. However, the method of FIG. 1C further includes additional steps including: (i) if no problems with the prescription are identified (Step 128), transmitting data regarding the prescription to be filled to an insurance company central database 110 to check (Step 134) for potential problems (drug interactions, early refills, insurance plan limitations, and so on). If no problems are identified (Step 136), then information indicating that the prescription may be dispensed is displayed (Step 138). However, if the records of the central insurance company database 110 indicate a problem fill problem (Step 140), then information displayed by the remote pharmacy terminal may indicate that the prescription should not be dispensed.

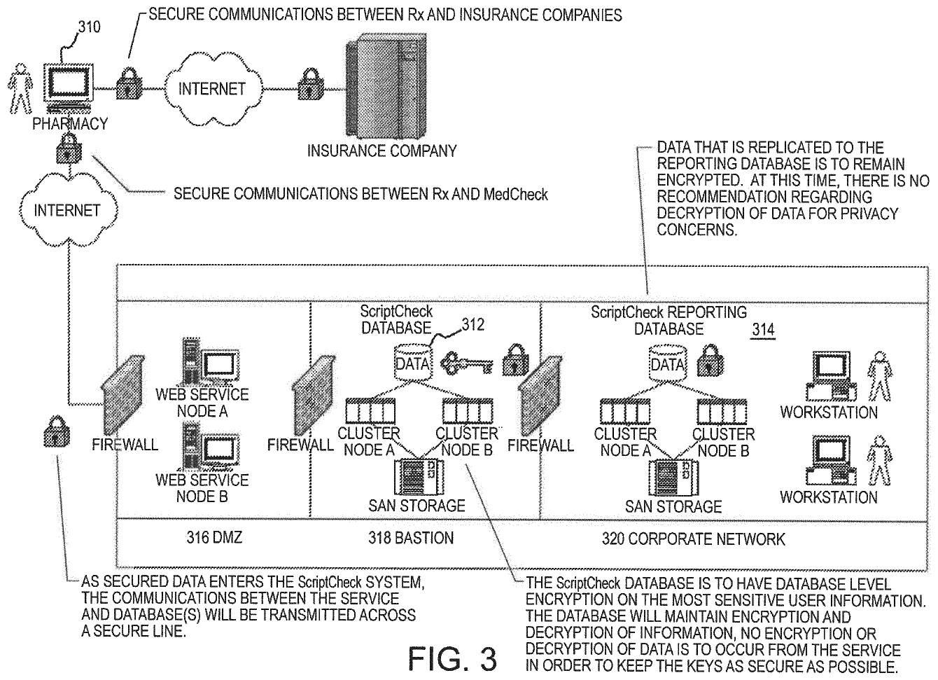

As shown, the central insurance company database 110 provides the advantage of receiving information from, and providing information to pharmacies from each of the three columns shown in FIG. 3. In other words, the central insurance company database 110 overcomes some of the limitations illustrated in FIG. 2, where a pharmacy in one column does not have access to information stored in a different database only utilizable to a different pharmacy in a different column. Unfortunately, the system and method described in connection with FIG. 1C still provides incomplete information to pharmacists that only works when the patient always uses the same universal insurance card.

Accordingly, a pharmacist using pharmacy terminal 102a may have access to information via insurance company database 110 that was not available in local database 104a, or retail chain central database 108. However, unfortunately, conventional system 100 leaves gaps in the information available for determining what information should be displayed to the pharmacist terminal requesting a prescription authorization. In particular, none of the conventional databases (104a, 108, 110) account for prescriptions filled outside the retail chain 106, in the case that no insurance identification information is presented, or, the case that the insurance card information is not of the particular insurance company associated with insurance company database 110.

FIG. 2 shows a server system 200 for medication monitoring-based prescription fill authorization checks in accordance with an exemplary embodiment of the present disclosure. In particular, FIG. 2 shows a universal prescription database server 408 that is configured to be universally-utilizable from any pharmacy, regardless of type, and regardless of whether a patient uses an insurance card or not. The dual direction arrows leading to and from the various pharmacy and insurance company devices of FIG. 2 and the universal prescription database server 208 illustrate the bi-directional communication capability via respective communication interfaces (e.g., FIG. 3) of the universal prescription database server. Preferably, the communication interface of the server 208 provides access to a wide area network with maximum availability, such as the Internet. In addition, the universal prescription database server 208 is preferably capable of exchanging prescription records and information with the insurance company central databases 110 and 406 (if authorized by the insurance company).

The universally-utilizable prescription database server 208 may also include a storage medium for storing various records as will be needed to perform the functions of the disclosure. For example, the records may include, in an exemplary embodiment, patient records including one or more unique patient identifiers (e.g., the patient's first and last name, date of birth, social security number), mailing address/phone number information, and prescription record history. Each prescription record entry may comprise one or more of: the unique patient identifier(s), a drug identifier (e.g., drug name and NDC number), and a quantity. The prescription records may also include a strength of the respective drug, instructions for use, day supply, the date the prescription was written, and a prescription fill date (if the prescription was filled). The prescription records may also include the prescriber's name (first and last), prescriber's DEA (Drug Enforcement Agency) number, prescriber's NPI number, prescriber's state license number(s), prescriber's full office address(es), office phone number(s) and office facsimile number(s). If the prescription of the prescription record has been filled, the prescription may indicate as much and also include information regarding the filled prescription including the pharmacy's name, full address, and phone number, and the pharmacist's full name, state license number, and NPI number. To achieve such, each prescription filled will be saved for future comparison (prescription authorization checks) and will indicate whether or not the user used a universally-accepted insurance card or not (e.g., how the prescription was paid). For example, if a patient uses a universally-accepted insurance card, the server 208 may capture, save, and transmit for pharmacist knowledge one or more pieces of the following: Insurance name, Insurance Bank Identification Number (BIN #), Processor Control Number (PCN #), prescription identification number (RX ID #), prescription group number (RX Group #), person code and insurance pharmacy help desk phone number. Subsequently, if no insurance card was utilized (e.g., cash/credit card was used), the prescription record will indicate that as well. With each transaction sent to the system, an updated list of each patient's drug allergies and health condition will occur and be stored in the systems database.

The universal prescription database server 208 of embodiments of the present disclosure provides several advantages over conventional systems and methods, as discussed above. In this respect, the universal prescription database server 208 preferably stores and transmits relevant prescription information for each individual prescription attempting to be filled, to and from pharmacies in order to provide pharmacists a complete list of any potential problems that exist. The universal prescription database server according to an embodiment of the present disclosure preferably incorporates a series of identifiers, or markers, which will be used to classify all medications according to the specific class into which they are classified. These classifications may include, for example, beta-blockers, opiates, or other classifications known as drug class identifiers. Additionally, the universal prescription database server according to an exemplary embodiment of the present disclosure may be preferably programmed to include an extensive list of drug interactions which will be used to determine if any interactions exist between any recently-filled prescriptions and the prescription currently attempting to be filled (the one at the basis of the prescription authorization request). The interactions are preferably classified according to severity. In one embodiment the lowest severity interaction may be classified as “(1),” and the most severe interaction may be classified as “(5)”. Additionally, and preferably, an exemplary system may use the drug class identifiers to determine if any medications were filled-recently, such as within the past 180 days, that would identify a duplicate therapy problem.

As shown in FIG. 2, the system 200 includes access to the retail pharmacy chain 106, which comprises individual retail pharmacies 102a-102e, and access to a central insurance company database 110. The system 400 may also (as depicted in FIG. 2) include a mail order pharmacy terminal 202, and an independent pharmacy 404, which could be the remote client pharmacy terminal. It should be appreciated that the selection of pharmacy types is meant to be illustrative only, and any combination of pharmacies and pharmacy types could be included. A second insurance company database 206 is also shown, representing that multiple independent insurance companies can maintain separate central insurance company databases, which may be accessed by pharmacies.

FIG. 3 shows a server system illustrating the flow of data from a user terminal 310 at a remote location (e.g., shown as a pharmacy, but could also be a hospital/doctor's office (prescriber)) through a series of secure communication interfaces to a server check database 312 back to the remote terminal 310. FIG. 3 shows how the exemplary embodiment may function. For example, when a prescription is presented to a pharmacy and entered into the pharmacy remote terminal 310 prior to being filled and dispensed, the data may be transmitted to a web service node terminal on an encrypted Secure Sockets Layer (SSL) connection. Once transmitted, the server system will communicate to the universal prescription database server on another encrypted SSL connection, which will be using a different certificate. Any data being returned to the client terminal 310 may be sent back across the same encrypted channels. The SSL connections will safeguard the data as it is in transit from the client to the host. Any patient protected information (PPI) that is stored in the universal prescription database is to be stored and encrypted as required by law (HIPAA). This data will not be decrypted to the reporting repository unless absolutely necessary (e.g., subpoena) and will follow strict policies and procedures to ensure that this data, whatever it may be, is secured to avoid any sensitive data being released from the system. The overall composition of the network begins with a perimeter network, also known as a demilitarized zone (DMZ). This network is protected on both ends by firewalls. This network adds an additional layer of security to the organizations local area network (LAN), making it least vulnerable to attacks. Within the DMZ is a sub-network identified as Bastion, also protected on both ends by firewalls. The Bastion provides another line of defense in the event of a security breach. The final network, the Corporate Network, is utilized primarily for day to day system maintenance as well as reporting capabilities. Access to this data will be at a corporate level only and will have an added firewall for protection.

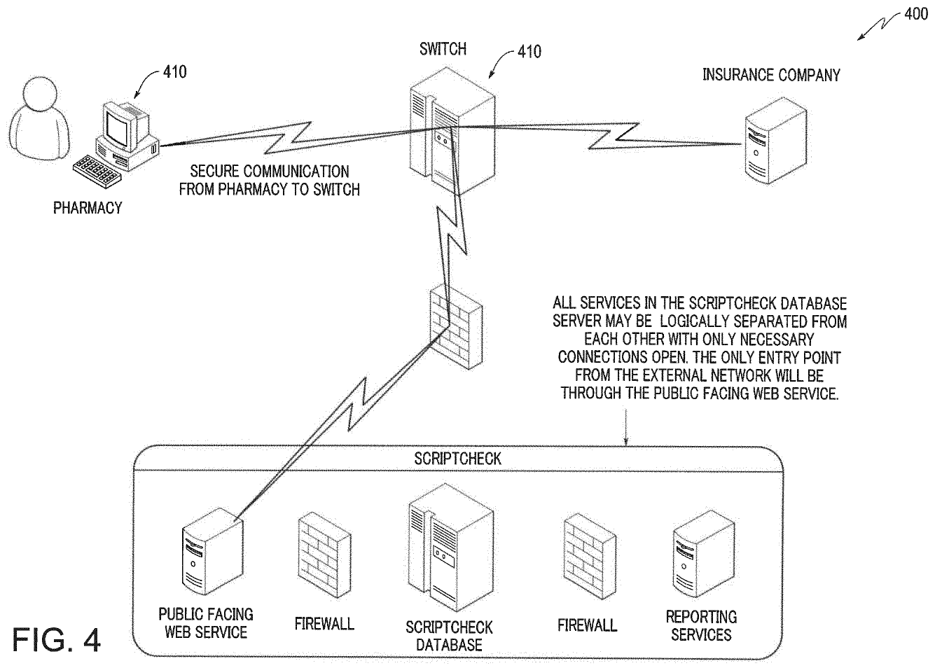

FIG. 4 shows another example of an embodiment of the disclosure. FIG. 4 shows a server system 400 illustrating the flow of data from a user terminal 410 at a remote location through a series of secure communication interfaces to a check server 412 back to the remote terminal 410. In this example, the first check is a hard drive of the pharmacy. If no problems, the pharmacy terminal communicates with a switch server 412, which communicates with a public facing web service server, preferably, with a firewall between the two. The switch server is configured to route the prescription fill request to destinations based on a BIN. The switch server 412 is the entity that reads the BIN number and routes the message via the BIN number. There are existing switch server companies (e.g., PowerLine, etc.) that route the fill/claim requests based on the BIN, and return information including, for example, an alert notification of a type of problem (e.g., refill too soon). However, in the conventional technology, people could not register to, for example, a retail chain database without using a pharmacy tied to that retail chain. Retail chains do not use a BIN number. By being able to provide utilizable database prescription information, without using the same pharmacy (e.g., instead by registering and receiving login credentials), any and every pharmacy could use the universal database to overcome problems with the existing technology. The public facing web service may communicate with a scriptcheck database server, which is universally-utilizable via the public facing web service. Ultimately, if there is no problem with any of the comprehensive series of checks, the prescription is auto-filled.

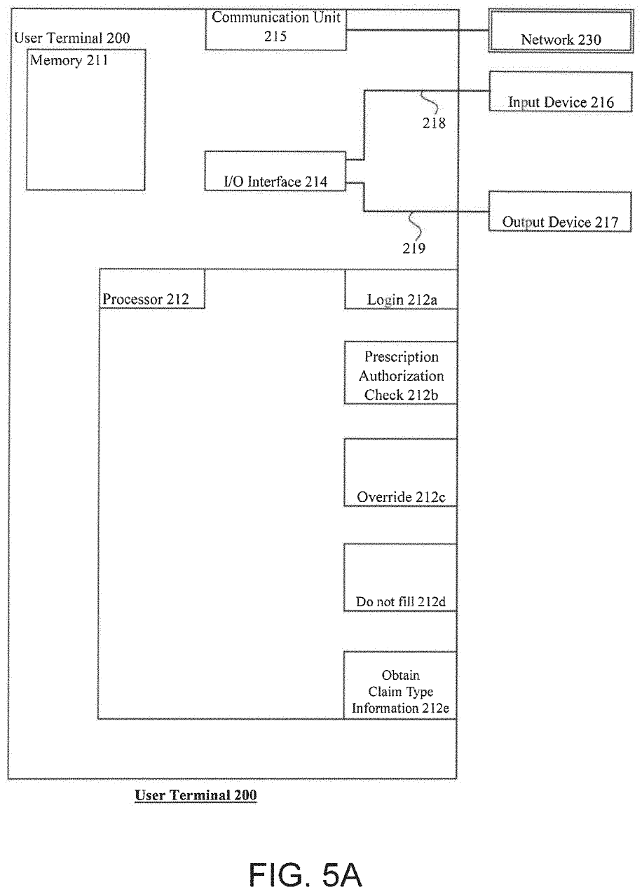

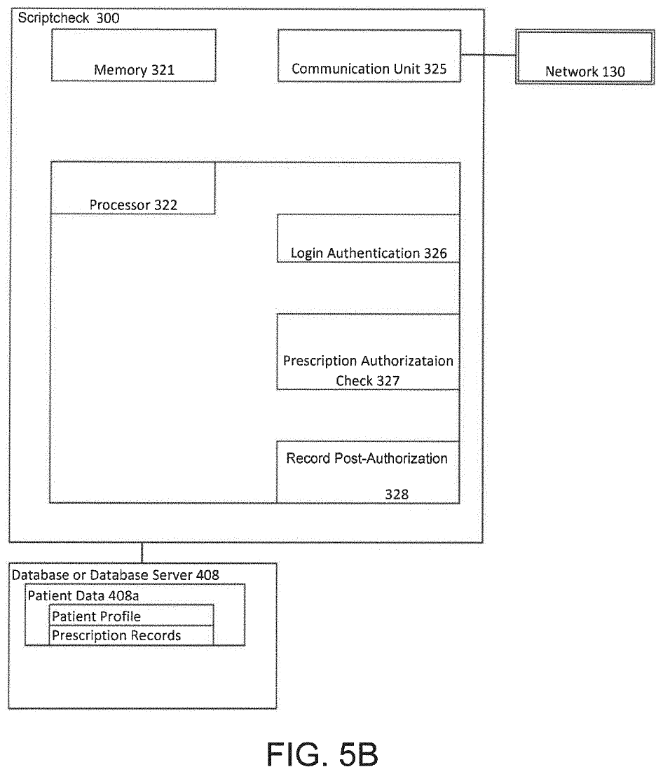

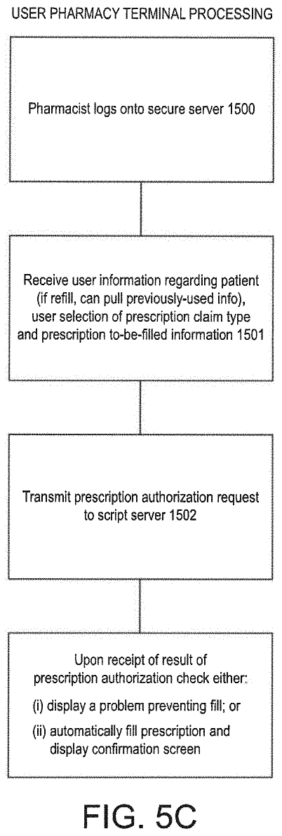

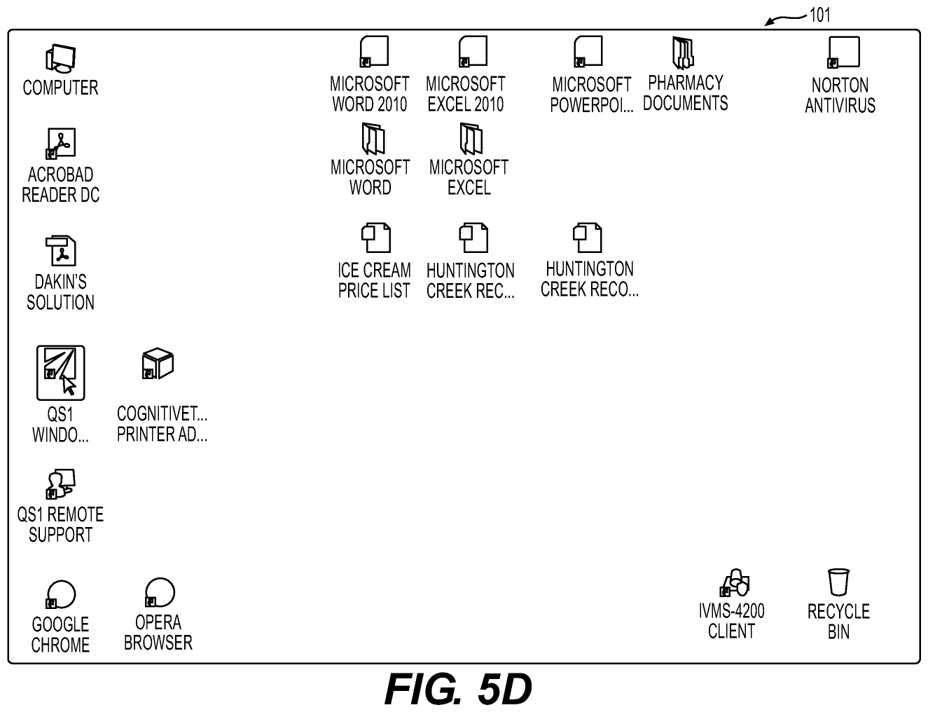

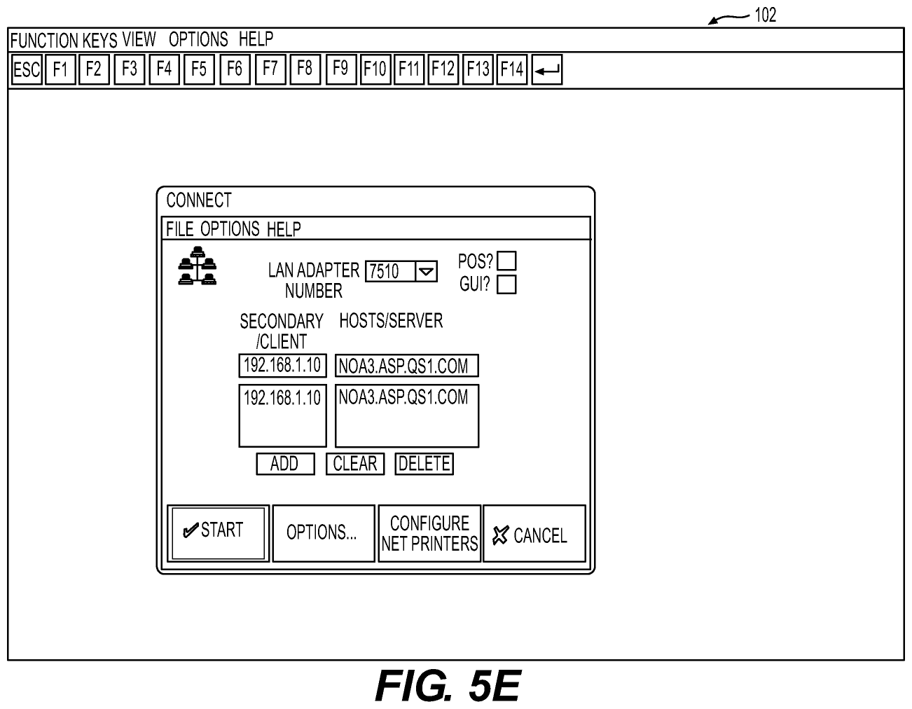

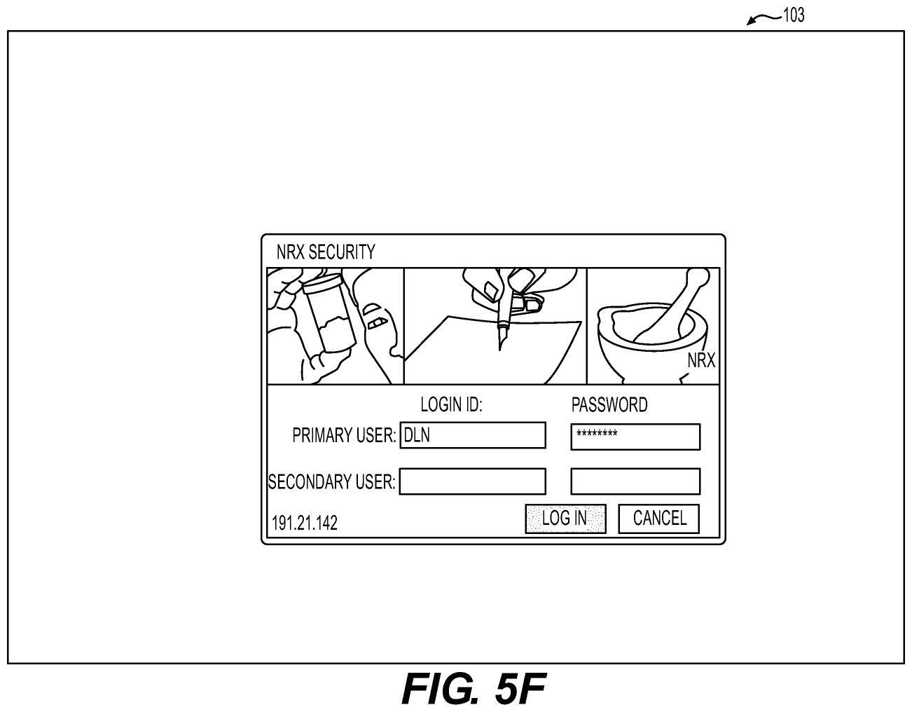

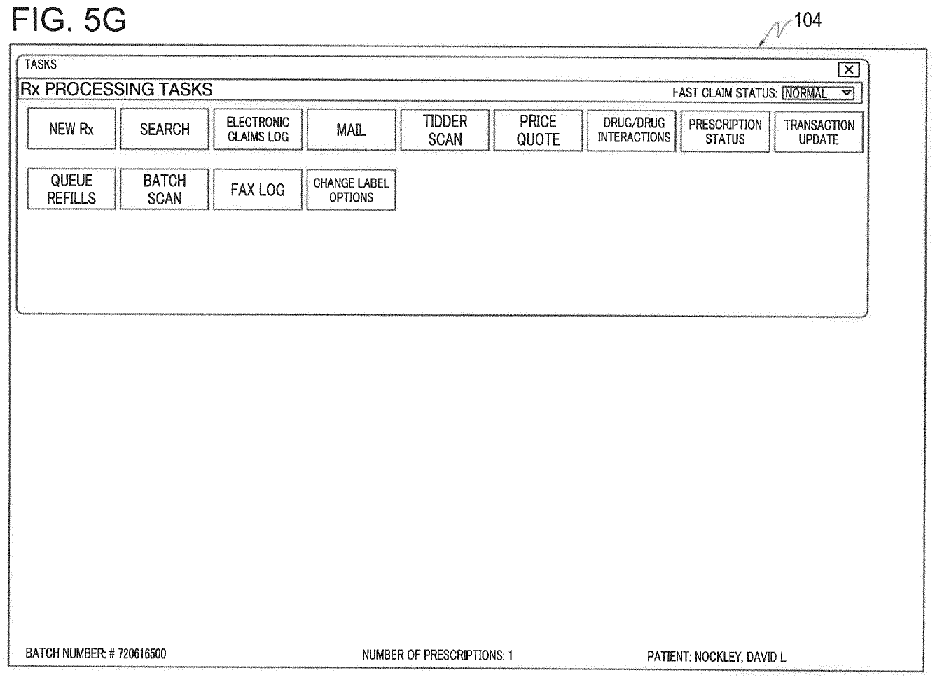

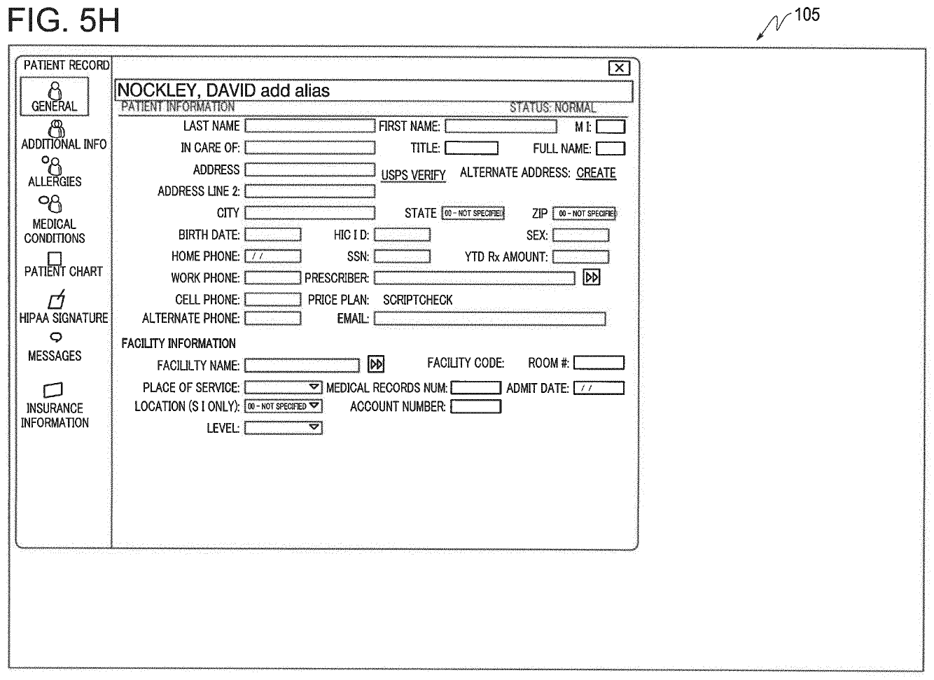

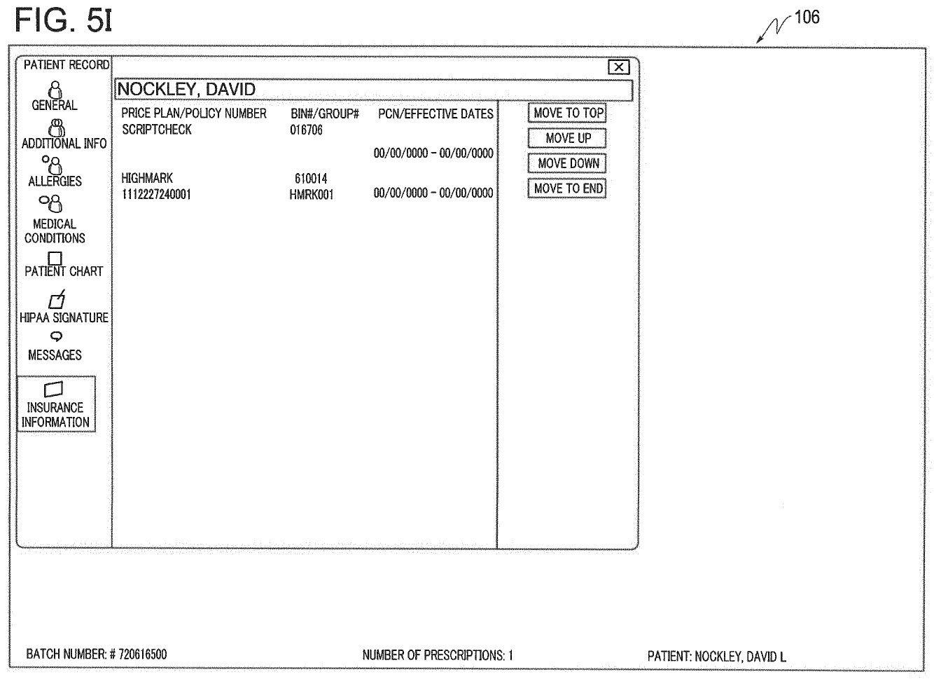

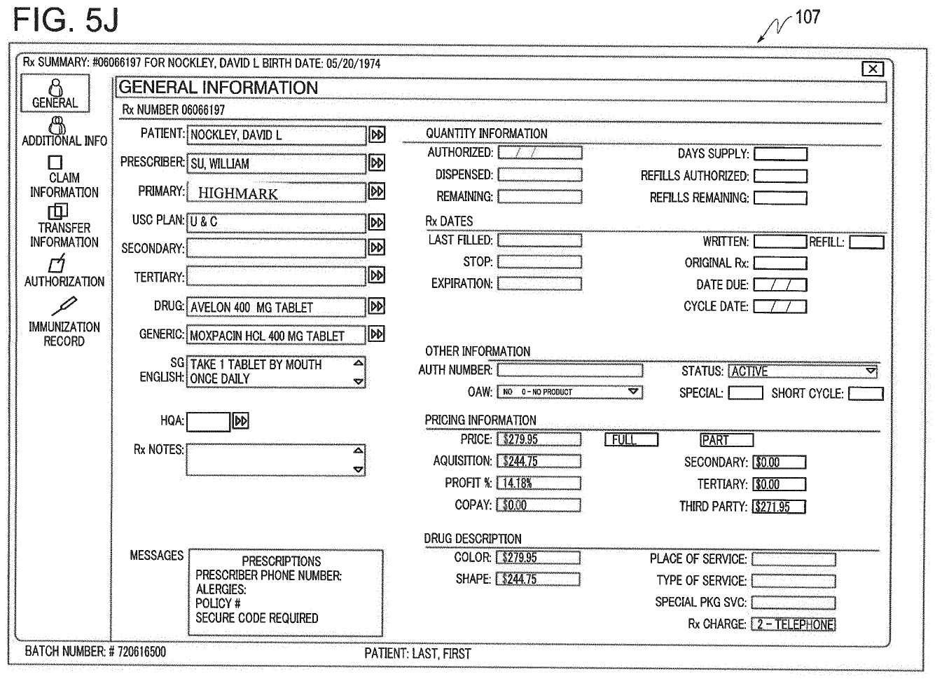

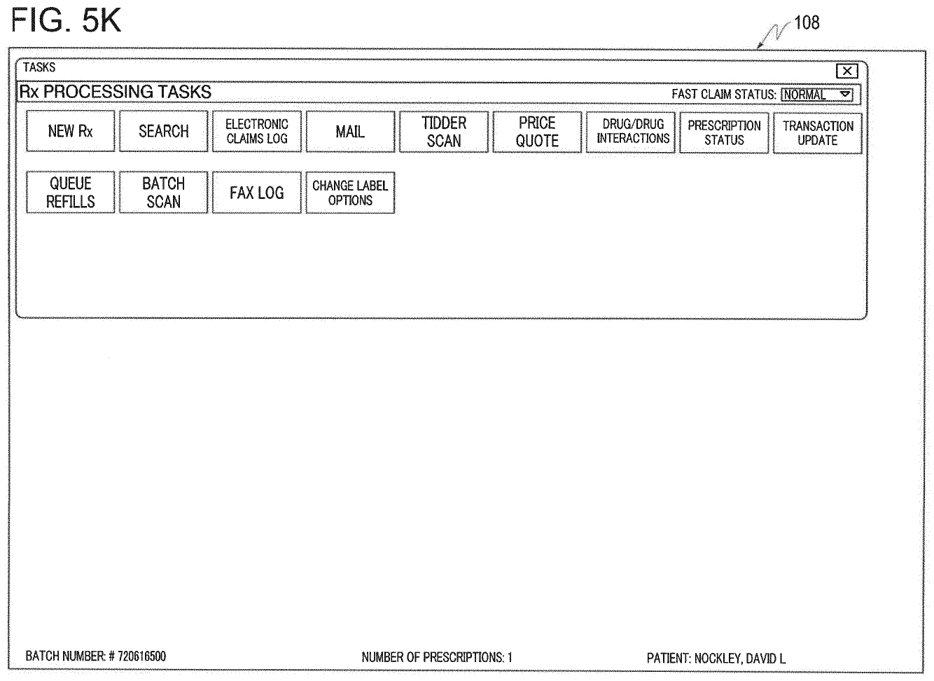

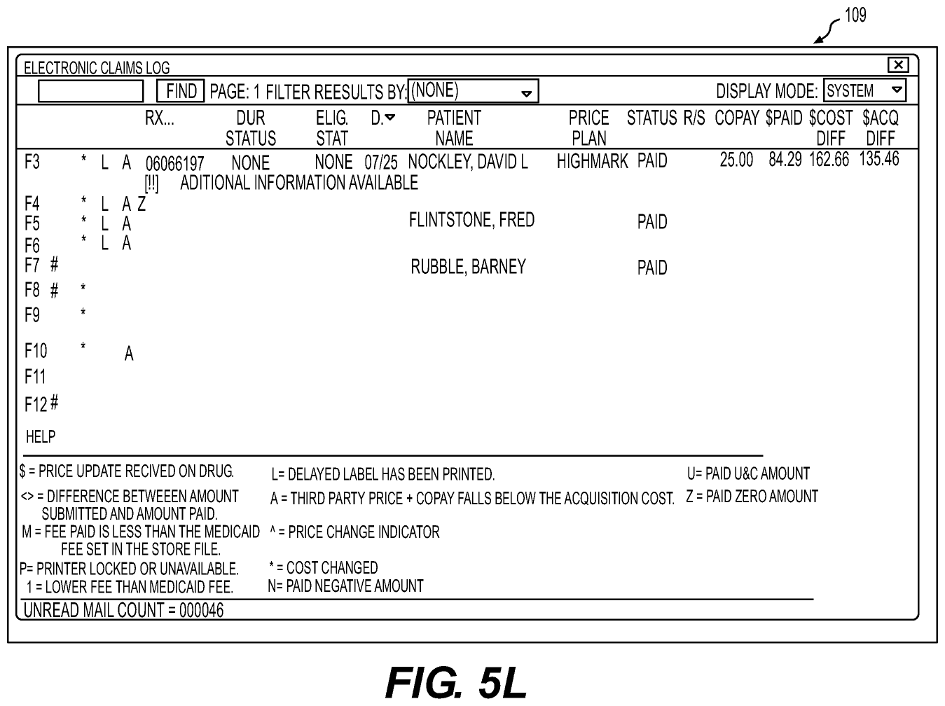

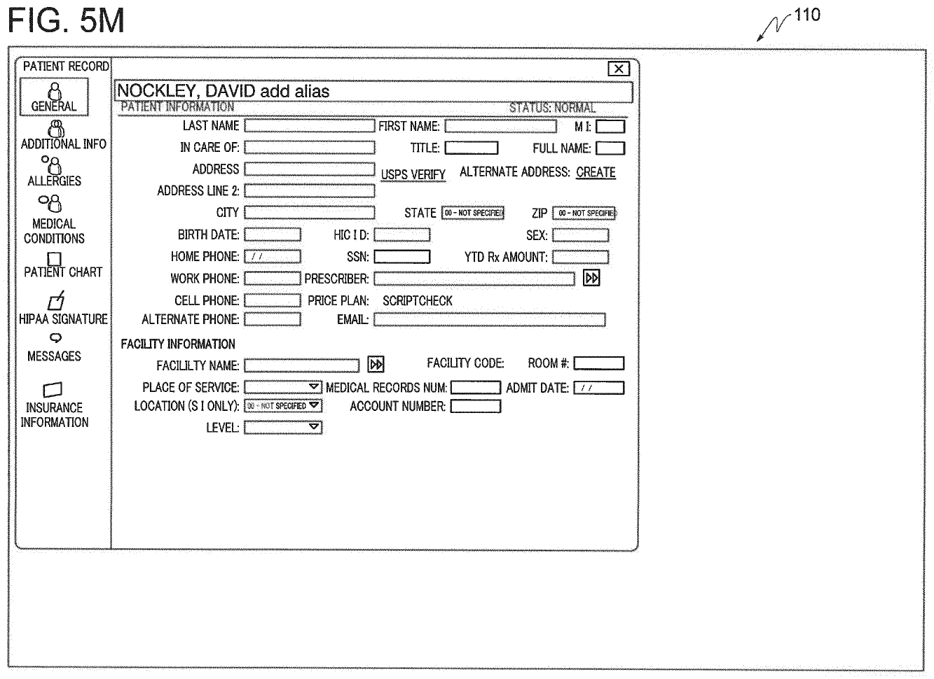

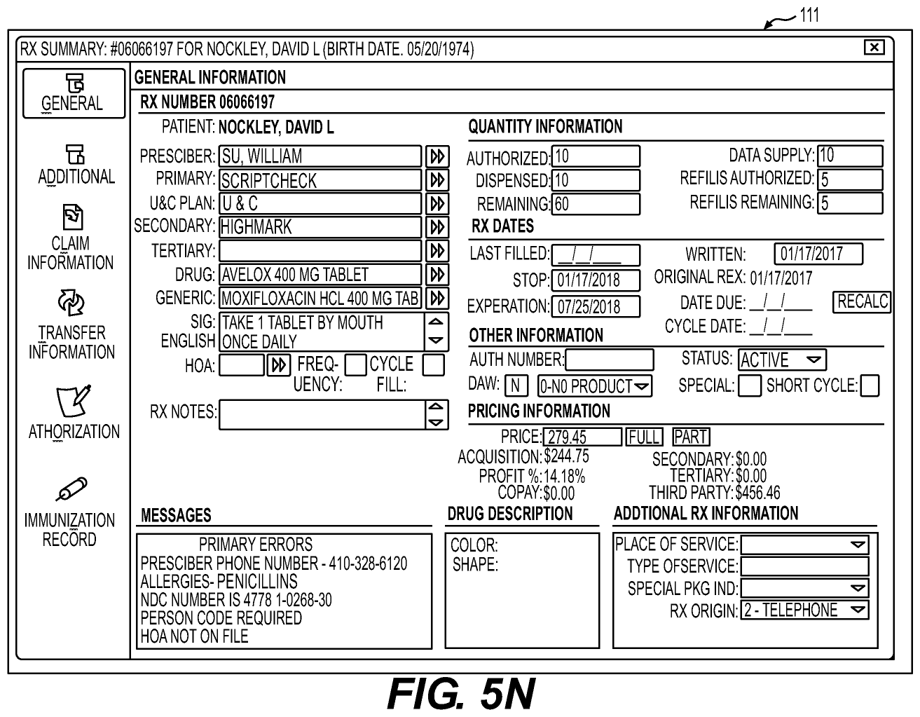

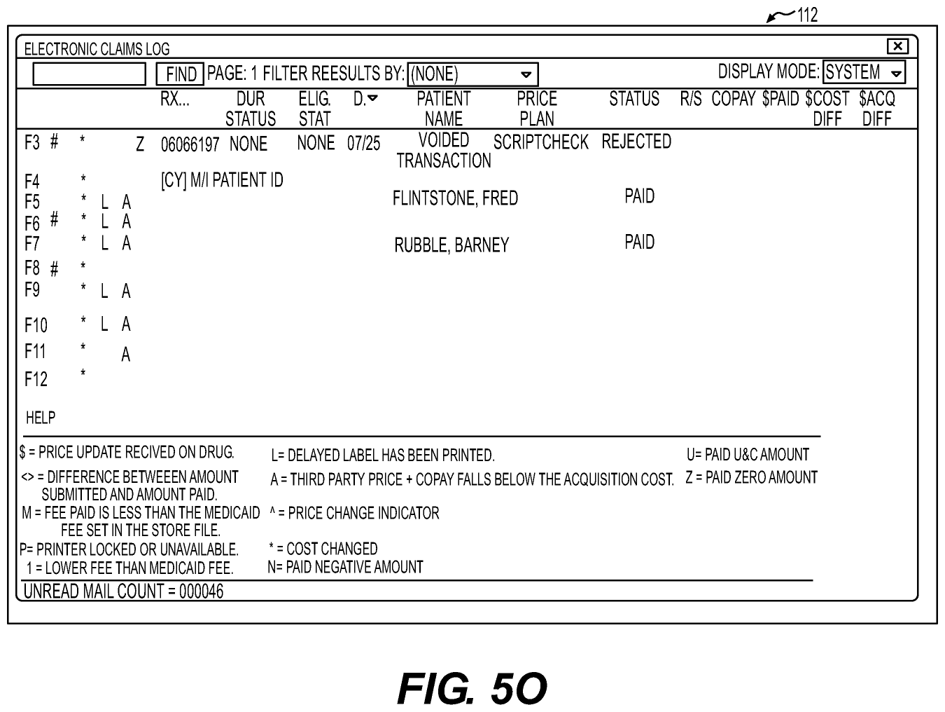

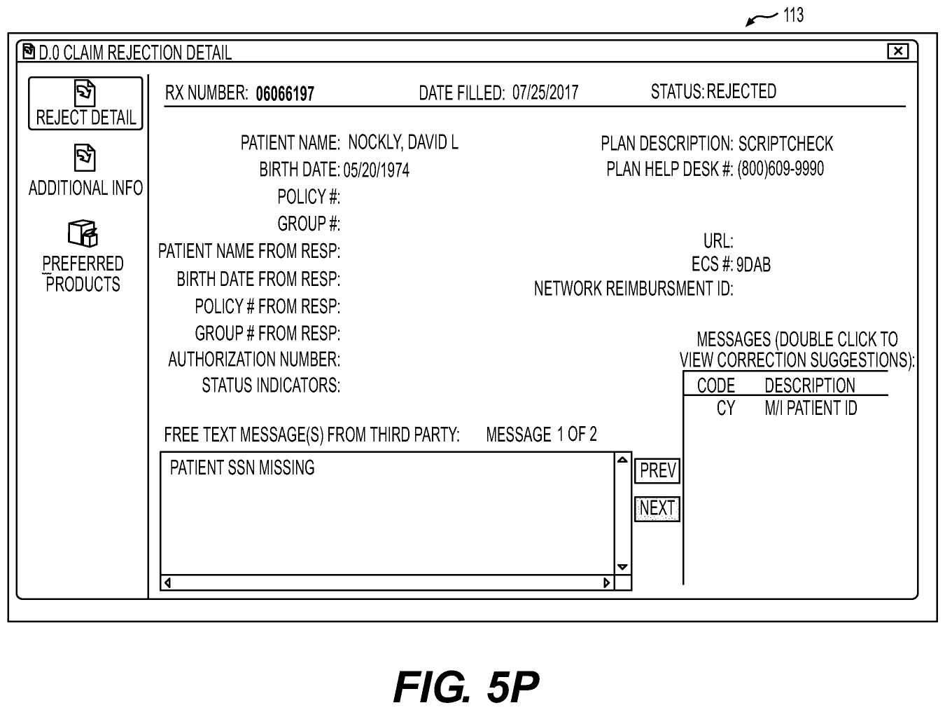

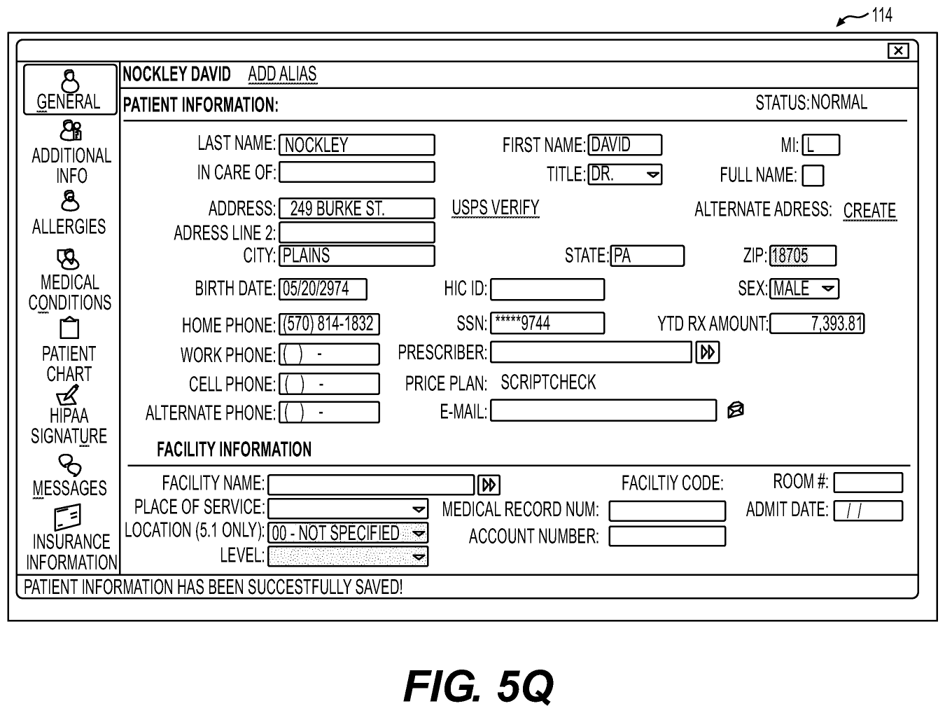

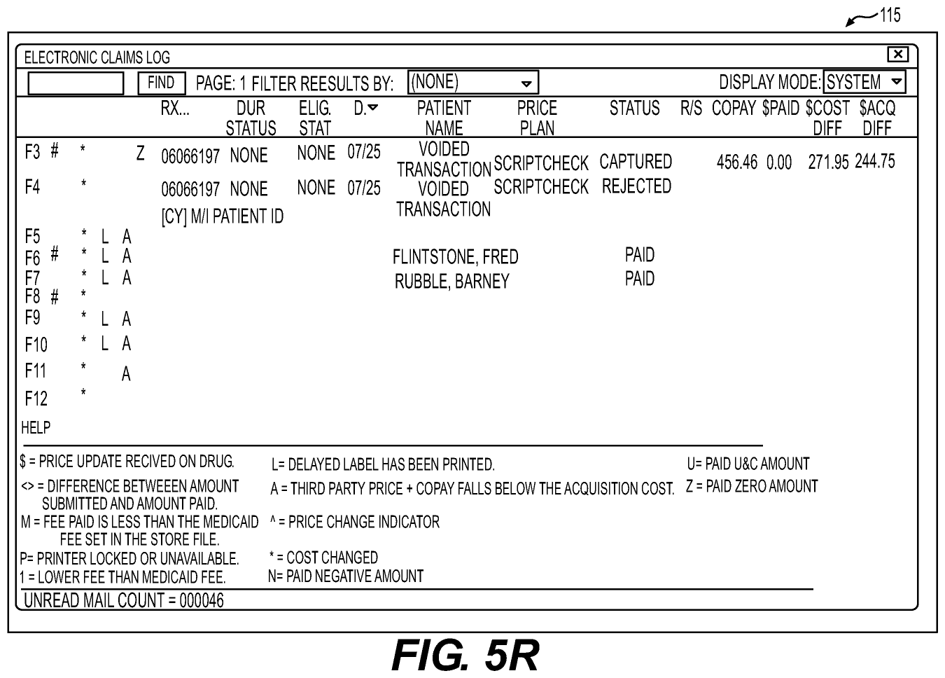

FIG. 5A shows the hardware of a user terminal 200, which may be located at a remote location. The user terminal device 200 may further comprise an Input/Output (I/O) interface 214 communicably coupled to an input device 216 and/or an output device 217 via links 218 and 219, respectively. The input device 216 may be one of or any combination of a keyboard, a mouse, a trackball, a touchscreen, a virtual reality glove, a sensor (e.g., biometric sensor), and any known or later-developed device for inputting data and/or control signals to the user terminal 200. The output device 217 may be one of or any combination of a computer monitor, a cathode ray tube, a liquid crystal display (LCD), a touchscreen display device, an image projector, an electrophoretic display, a virtual reality device, an audio speaker, and any other known or later-developed device for visually displaying or audibly outputting the data output from the user terminal 200. Each of the various links 218 and 219 can be any known or later-developed device or system for connecting the input device 216 and the output device 217, respectively, to the I/O interface 214. In particular, the links 218 and 219 can each be implemented as one or more of a direct cable connection, a connection over a wide area network, a local area network or a storage area network, a connection over an intranet, a connection over an extranet, a connection over the Internet, a connection over any other distributed processing network or system, and/or an infrared, radio-frequency or other wireless connection.

The user terminal 200 may further comprise a communication unit 215 (which may comprise a network interface card) that is communicably coupled to the network 230 and that allows the user terminal 200 to communicate, via the network 230, with the scriptcheck universally accessible server 300.

The user terminal 200 may also include various circuits, routines, or applications as will be discussed below with reference to items 212a-212c. These disclosed circuits, routines, or applications can be dedicated circuits and/or individual programs and/or routines in a larger program stored in a memory such as the memory 211 and executed by the processor 212. Thus, in exemplary embodiments, the disclosed circuits, routines, or applications can be implemented by one or more programs executed by the processor 212. In certain embodiments, circuits, routines, or applications may be a web browser or application, and will be discussed in more detail below.

FIG. 5B shows an authorization check server 520 and a profile database 540 of the embodiments of anyone of FIGS. 2-4. FIGS. 5A and 5B show modules 212a-212c and 326-328, respectively, which may perform some of the functions of the user terminal in communication with the server 300.

In this respect, FIG. 5C further shows a high-level flowchart showing some of the processing executed by the user terminal in communication with the server 300 according to an exemplary embodiment of the disclosure for performing processing for a pharmacy location (e.g., a brick-and-mortar independent pharmacy). In particular, FIG. 5C shows steps 1500-1504. In particular, FIGS. 5D-5L are further described below to explain Steps 1500-1504, and additional processing interactions between the client terminal 200 and the server 300 that may be implemented in different embodiments of the disclosure.

FIG. 5D shows a screenshot of a display (output device 217) of the user terminal 200 showing a “highlighted” icon with an “arrow” over it, which is the icon to start the process. The user of the user terminal may click a mouse on an icon to start the process, or, if on a smartphone, touch the screen. The icon may be an icon on a desktop background screen for example.

FIG. 5E shows a screenshot of a display (output device 217) of the user terminal 200 displaying how to login, which is to authenticate the user via the login function 212a. Thus, FIG. 5E corresponds to a display that is a part of the Step 1500 where the user (e.g., a pharmacist) logins to the secure server. To login to the secure server, the user may have registered in advance via the public facing web server so that the user can use the system and so that the login credentials are stored. The login credentials may be a user name and password, or other types of authentication processing, such as, biometrics (e.g., iris scan, fingerprint) or an auto-login feature where the password is remembered. However, unlike conventional retail systems, the user is able to gain access to the system by registering with the server system. Similarly, FIG. 5F shows a screenshot of a display (output device 217) of the user terminal 200 displaying where to enter login credentials. FIG. 5G shows a screenshot of a display (output device 217) of the user terminal 200 displaying, once login has been authenticated, an Rx Processing Tasks Screen 104 which includes multiple buttons for the pharmacist. For example, a search button, which will be described later for searching for a patient. Once the user clicks the search button, the user may look up a patient via a search bar.

FIG. 5H shows a screenshot of a display (output device 217) of the user terminal 200 displaying the patient's profile. In this screenshot, the user's SSN has not been entered. This screen 105 would be a part of the Step 1501, which requires 3 types of information. For example, to start the process, a user (e.g., pharmacist) must enter user information regarding a patient (if a refill, could use previously stored information to enter), and information regarding the prescription claim type (e.g., a name of a nationally recognized insurance carrier who would have a BIN discussed below, or an indication NONE or CASH, which mean that no nationally recognized insurance card information is presented for this prescription claim.

FIG. 5I shows a screenshot of a display (output device 217) of the user terminal 200 displaying a Claim Type Information screen 106. In this example, the patient has insurance so the patient's insurance information that is on file (which is via the “insurance information” tab on the bottom left (highlighted) is displayed).

FIG. 5J shows a screenshot of a display (output device 217) of the user terminal 200 showing a display screen 107. The display screen 107 is used to prompt the pharmacist to enter a prescription for filling. In this example, as the claim types (primary, secondary) do NOT use the disclosed universally-utilizable system, they are less accurate. PLEASE NOTE***IN THIS EXAMPLE, THIS PRESCRIPTION IS BEING SUBMITTED ONLY TO THE PATIENT'S PRIMARY INSURANCE (HIGHMARK)*** This would be an example that is similar to how the current, conventional system works that is inaccurate/incomplete information because DOES NOT use the disclosed system.

FIG. 5K shows a screenshot of a display (output device 217) of the user terminal 200 displaying, once login has been authenticated, an Rx Processing Tasks Screen 104 including a search button. From this screen 108, the pharmacist selects the “electronic claims log” to see if the prescription processed with or without errors.

FIG. 5L shows a screenshot of a display (output device 217) of the user terminal 200 displaying, an Electronic Claims Log screen 109. Please note that this prescription (rx#6066197, top on the list) processed without any errors to the insurance company and resulted in a “paid” claim. It is important to note that this claim was submitted WITHOUT the patients social security number. ***OUR SYSTEM REQUIRES A SOCIAL SECURITY NUMBER (AMONG OTHER THINGS) TO ENSURE THAT WE HAVE THE CORRECT PATIENT***

FIG. 5M is similar to FIG. 5H and shows a screenshot of a display (output device 217) of the user terminal 200 displaying the patient's profile. This screen 110 occurs after selecting “search,” and a patient's name is typed in the search bar, and a result is found (and optionally selected). FIG. 5N shows a screenshot of a display (output device 217) of the user terminal 200 showing how a pharmacist enters a prescription for filling using an embodiment of the disclosed system. In FIG. 5N, as shown in Screen 111, the primary form of prescription authorization is SCRIPTCHECK, which corresponds to using a universally-utilizable server as in the disclosure. That is, FIG. 5N IS using our system. PLEASE NOTE***IN THIS EXAMPLE, WHEN THE USER OF THE USER TERMINAL CLICKS THE FILL BUTTON, THIS PRESCRIPTION IS SUBMITTED TO THE DISCLOSED SYSTEM (LOCAL HARDDRIVE and SCRIPTCHECK) FIRST, THEN TO THE PATIENT'S PRIMARY INSURANCE (HIGHMARK)*** This is seen under the “primary” section, which shows SCRIPTCHECK and under the “secondary” section, which shows HIGHMARK (the patient's primary insurance). In this respect, when the FILL button is selected/clicked, the prescription information and patient information (i.e., identification information) goes to the internet to the switch server, and then routed to the universal database server (and if insurance the insurance server). If neither have problems, in an embodiment, the user terminal automatically issues a command to print a prescription label for the identified patient with the prescription information. Further, a confirmation screen may be displayed confirming that the prescription is filled/dispensed.

One of the important things to note is that using the disclosed system adds NO ADDITIONAL STEPS to the prescription filling process. This is seen in the FIGS. above. In this respect, the disclosed system may be implemented to seamlessly integrate (“blindly drop”) into all pharmacy prescription processing systems. This would mean that no additional steps need to be taken by the pharmacist to ensure that EVERY claim, including those that aren't being processed to an insurance company, go to the disclosed universally-utilizable database (SCRIPTCHECK) first.

FIG. 5O shows a screenshot of a display (output device 217) of the user terminal 200 showing a rejection from the universally-utilizable server system. In particular, FIG. 5O shows a main Electronic Claim Log screen 112 including an indication that RX #6066197 has a “VOIDED TRANSACTION.

FIG. 5P shows a respective screenshot of a display (output device 217) of the user terminal 200 showing a rejection from the universally-utilizable server system. In particular, FIG. 5P shows a screenshot of a Claim Rejection Detail Screen for RX #06066197. The Claim Rejection Detail Screen indicating to the pharmacist that the patient's social security number is missing and MUST be entered in the patient profile, and the claim re-submitted.

FIG. 5Q shows a screenshot of a display (output device 217) of the user terminal 200 showing the patient profile with the patient's social security number entered on their profile. In some embodiments, SCRIPTCHECK may be configured so that a SSN is required for every transaction. This ensures proper verification of the patient. In some embodiments, the personal identification information includes a first and last name and a date of birth with the SSN.

FIG. 5R shows a screenshot of a display (output device 217) of the user terminal 200 showing the Electronic Claims Log screen showing the claim for rx#6066197 processed through SCRIPTCHECK successfully. That is, after the user of the user terminal enters in the SSN in FIG. 5Q then the SCRIPTCHECK problem goes away and the prescription can be filled and dispensed.

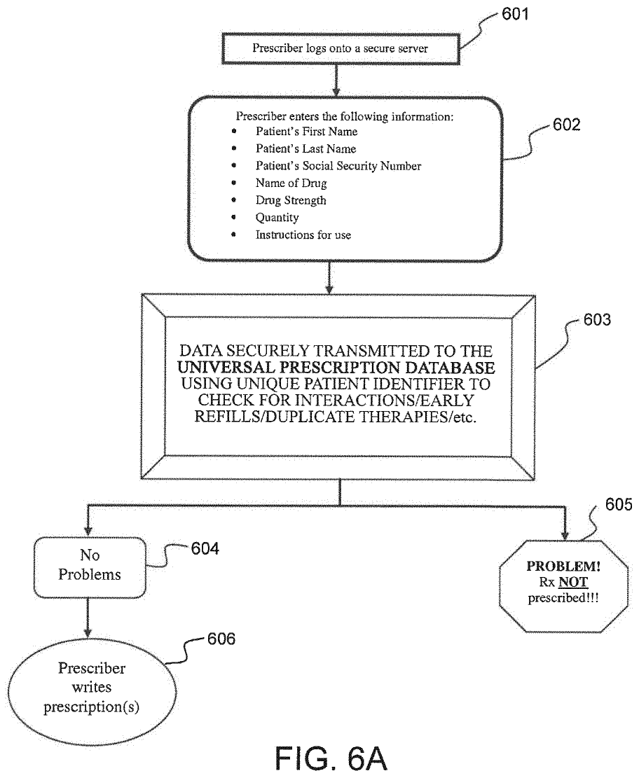

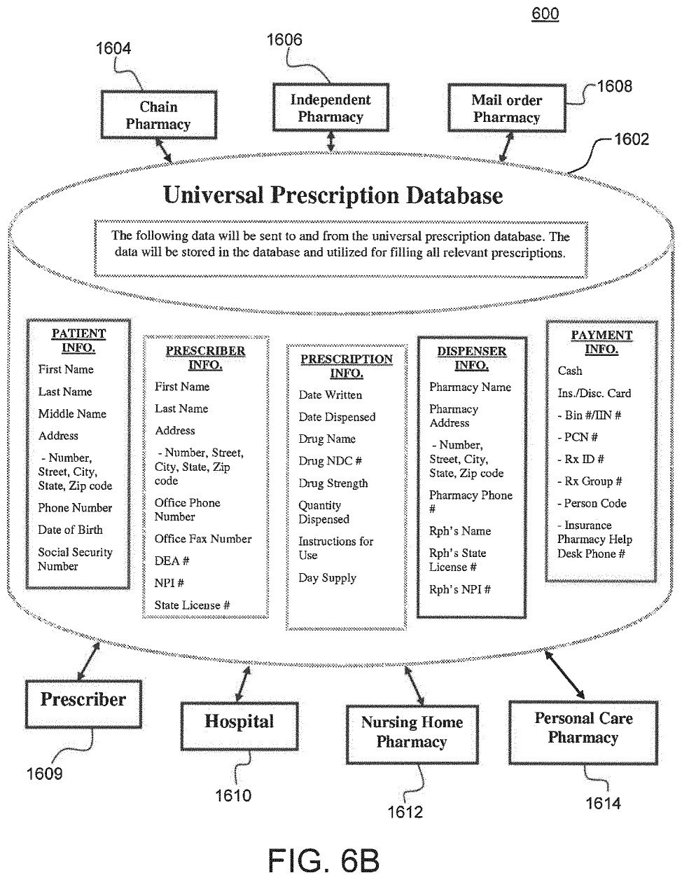

FIG. 6A shows a high-level flowchart showing some of the processing executed by an embodiment of the discloser related to a terminal device of a prescriber utilizing a universally-utilizable database server according to an exemplary embodiment. In this respect, the method of FIG. 6A may be used by a prescriber to receive displayed information that helps check for any problems in prescribing a specific prescription before issuing the prescription to the patient. FIG. 6A illustrates use by a prescriber, however, as discussed above and below, a pharmacist may use the system as well. When a prescriber decides to write a prescription, the prescriber will be able to utilize the most current, up-to-date data for the patient. After logging into a secure network (Step 601), the prescriber enters the patient information and prescription information (e.g., a patient's demographically data as well as the full prescription data for the designated prescription) at Step 602. Upon transmitting the claim to the universal prescription database server across the secure network (Step 603), a comprehensive check will be performed to check for problems with filling the prescribing the proposed prescription (e.g., problems related to one of: drug allergies, negative drug-disease state interactions, negative drug-drug interactions, duplicate therapies, early refills (overuse of a medication), and other potential negative problems). In real-time, a response will be transmitted back to the remote terminal informing the prescriber of any potential problems) at Step 605. It is important to note that this data will NOT be stored in the universal prescription database server. It will only be used for the prescriber to see what the patient already has had filled and by which prescriber(s). Only once the prescription is actually filled and dispensed will the data be updated in the centralized system and made available for future reference.

FIG. 6B shows an exemplary embodiment of a universal prescription database including attributes or characteristics of medication data (including prescriptions and disbursements each including associated patient info) stored in the universal prescription database, and communication interfaces between a plurality of devices. The system 600 of FIG. 6 includes a universal prescription database server 1602 that is connected to a wide variety of pharmacies as well as being utilizable by all licensed prescribers. As depicted, the pharmacies connected to universal prescription database 1602 include a retail chain pharmacy 1604, an independent pharmacy 1606, a mail order pharmacy 1608, a hospital 1610, a nursing home 1612, and a personal care facility 1614. Of course, those of ordinary skill in the art will readily appreciate that the particular pharmacy types depicted in FIG. 6 are merely exemplary, and intended to illustrate the wide variety of pharmacies which may participate in the universal prescription database server 1602. As shown, each pharmacy is capable of communicating with the universal prescription database server 1602 and exchanging information in both directions. New prescription records are transmitted to the universal prescription database server 602. Preferably, for each relevant prescription, the following information may form a prescription record that is transmitted to the universal prescription database 1602 and utilized for future prescription checks: Patient data (e.g., one or more of First and Last name, middle name, full address, phone number(s), date of birth, social security number), Prescriber data (e.g., one or more of: First and Last name, full office address, office phone number(s), office facsimile number(s), DEA #, NPI #, state license number(s)), Prescription data (e.g., one or more of: Date written, date dispensed, drug name, NDC #, drug strength, quantity dispensed, instructions for use, day supply), and, if applicable Dispensing data (e.g., one or more of: Pharmacy name, full address, phone number, dispensing pharmacist's first name, dispensing pharmacist's last name, dispensing pharmacist's state license number(s), dispensing pharmacist's NPI #), and claim type (e.g., what, if any, insurance claim information is used with submitting the prescription claim, and if none, then an indication of NONE. The term CASH may be used to describe the situation where a user is not submitting insurance claim information with a prescription fill request. The insurance or discount card information for the claim may include one or more of: BIN #, PCN #RxID #, Rx Group #, Person code, Insurance's/discount card's pharmacy help desk phone number). In addition, it is advantageous for each record to be associated with a patient name and a unique patient identifier, such as a social security number. It should also be understood that licensed prescribers will also have access to the same information via an equally secure network to help in determining whether or not to prescribe a prescription(s). Generally speaking, as shown in FIG. 6B, some information may be required to submit a claim. For example, in an embodiment, a SSN may be required to submit a prescription fill request. In other embodiments, a First and Last Name, DOB and SSN is mandatory in order to recreate a patient profile, or at least, for every fill request. Obviously, people's addresses change, thus these generally are not good for personal identification information.

In this respect, the method may include The method further includes receiving a database request via a Bank Identification Number (BIN) specific for the comprehensive prescription database from remote terminals via a secure/encrypted internet communications interface of the universal prescription database. The database request comprises of a new prescription or a refill of an existing prescription. The method includes comparing the transmitted prescription record using the industry standard NCPDP format with existing prescription records associated with the same unique patient identifier and preparing a response based on the comparison in real time. The method further includes sending the response to the remote terminal via the secure communications interface. The responses include, but are not limited to, drug allergies, negative drug-disease state interactions, negative drug-drug interactions, duplicate therapies, early refills (overuse of a medication), and other potential negative problems, in essence providing a comprehensive pre-dispensing check of each prescription filled.

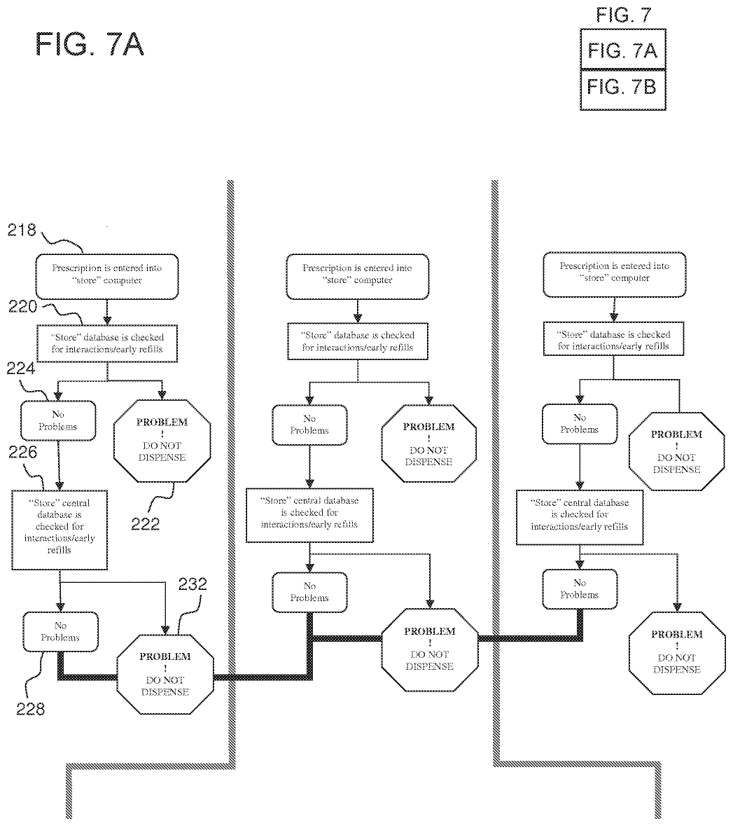

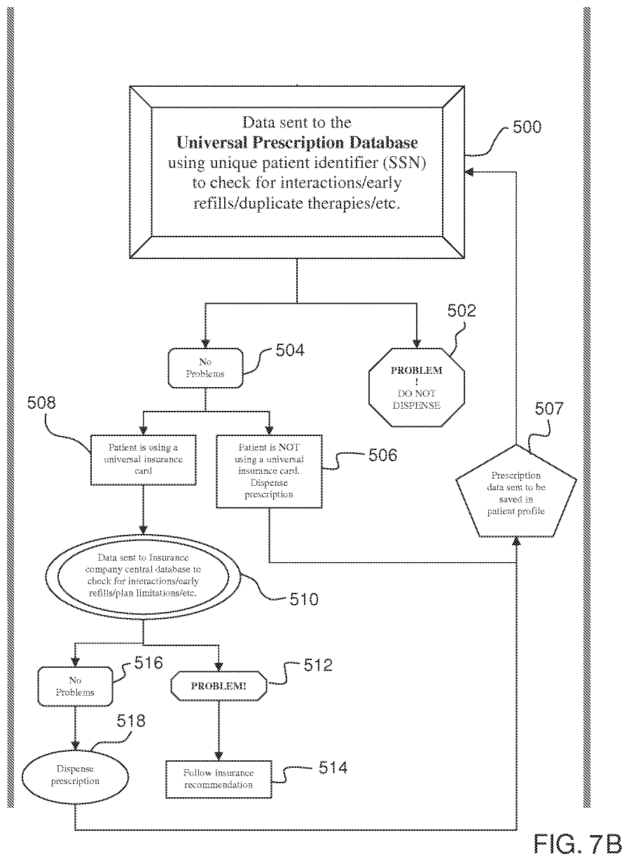

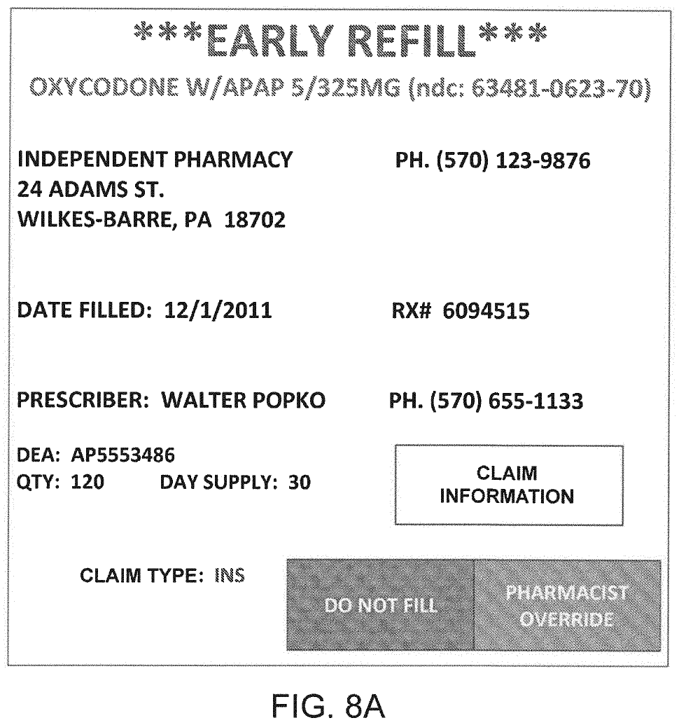

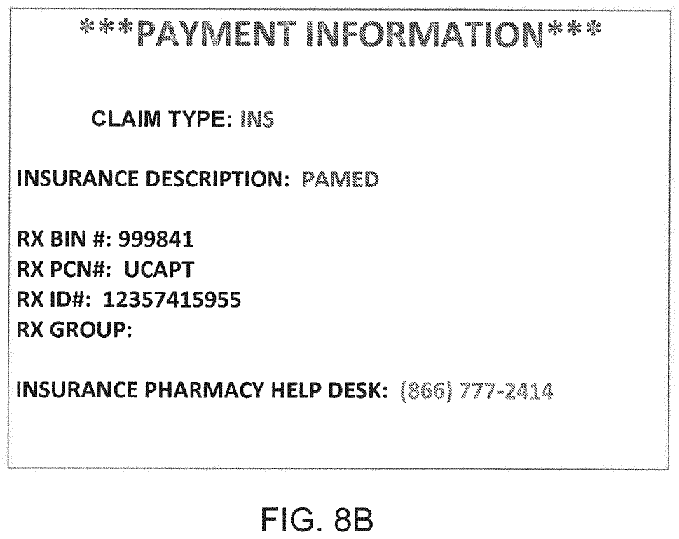

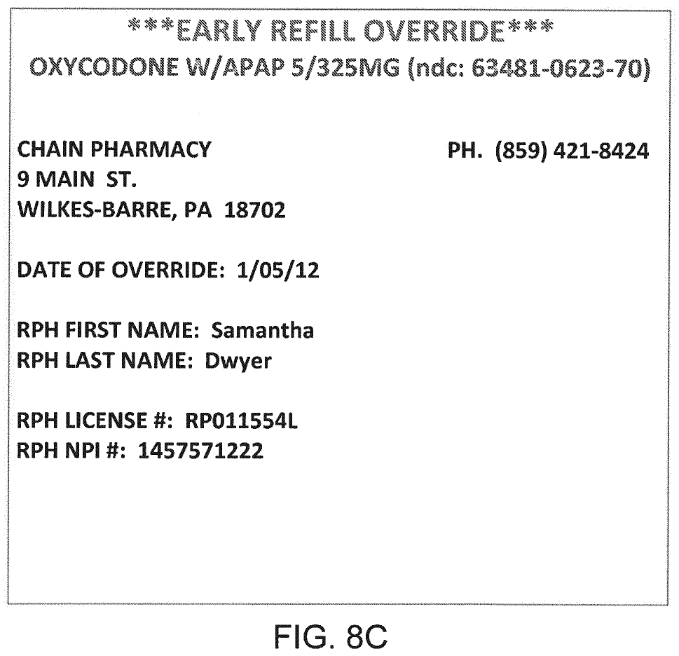

A method of filling a prescription according to an exemplary embodiment of the present disclosure will now be described in connection with FIGS. 7A and 7B. The first portion of the process, from a prescriber writing a prescription (Step 200) through the pharmacy central database being checked (Steps 226, 228, 232) are substantially the same as described in connection with FIG. 2, and so a detailed description thereof need not be repeated. If the check of the local database and the central database both return positive results (in FIG. 7A), then according to exemplary embodiments of the present disclosure, the responsive server transmits the prescription information to a universal prescription database server at step 500 in FIG. 7B. Preferably, the data transmitted to the universally-utilizable prescription database includes a unique patient identifier, such as a social security number. The universal prescription database receives and transmits data relevant to determining whether each respective prescription attempting to be filled should be filled at any pharmacy terminal regardless of sector (retail, hospital, mail order, among others), and regardless of prescription type, e.g., whether insurance card information or not (e.g., cash payment), or otherwise. Accordingly, the universally-utilizable prescription database server advantageously has the most complete prescription history information available for each patient, regardless of which pharmacies, or how many pharmacies they have used, and regardless of the manner in which the patient pays for their prescriptions, past or present. If the universally-utilizable prescription database server identifies a potential problem, that information is transmitted back to the pharmacist (see FIGS. 8A-13), who can make a decision not to dispense, at step 502 (e.g., by selecting the “DO NOT FILL” button in FIG. 8A as an example). If the universal prescription database server does not identify any potential problems, than that status is transmitted to the pharmacist at step 504. At this point, the pharmacist has confidence that the prescription can be filled with a minimum chance of medication being dispensed improperly due to limited information available to the pharmacist.

The universally-utilizable database server may also perform an insurance check in steps 510-518, if insurance information is presented. However, this insurance check could be performed before or after the universally-utilizable database stored information is checked. Further, the universally-utilizable database server may be one server or incorporate additional servers. For example, as described above, an intermediary scriptcheck server may be used to securely communicate with the universally-utilizable database server. However, one server could perform both functions. In this respect, if once the universally-utilizable prescription database has been utilized, if the patient is not using a prescription insurance card, the process can stop, after the prescription is dispensed (Step 506), and the dispensed prescription data is transmitted to be saved in the patient profile (Step 507). If patient insurance card information has been entered into the remote terminal (Step 508), then a record of the prescription request can be transmitted to the insurance company central database 110 server (Step 510). The information transmitted to the insurance company database server 110 can preferably include the results of the universal prescription database check. The insurance company may still identify a problem, such as ineligibility for the particular medication under the patient's insurance coverage, among other potential problems, as will be understood by those of ordinary skill in the art. Accordingly, the insurance company database 110 may alert the pharmacist to a problem (Step 512). The insurance company's recommendation may be followed (Step 514). However, if the insurance company database server 110 does not identify any problems (Step 516), then the pharmacist may dispense the prescription (Step 518). Preferably, the result of the pharmacist ultimately filling a prescription is then transmitted to the universal prescription database so that the universal prescription database has the most complete set of prescription history data available.

Of course it should be appreciated that the above described system and method are merely exemplary and various changes to the system and method described above may be made without departing from the scope and spirit of the invention. For example, a particular insurance company may determine that the universal prescription database is superior and more cost effective than continuing to maintain their own separate database 110. Accordingly, particular insurance companies may solely utilize the universal prescription database. As such, the universal prescription database could be programmed to analyze eligibility rules of the particular insurance company according to the insurance company's policies. Similarly, individual pharmacies and retail chains may eventually forego maintaining separate prescription databases in favor of the universal prescription database described herein.

As will be understood, utilizing an exemplary system and/or method as described above, each time a prescription is written by a prescriber and filled at any pharmacy that utilizes a universal prescription database, an extensive search of all available prescription records will be performed to identify potential problems regardless of how many different prescribers and/or pharmacies the patient has used in the past. Advantageously, the results will include not only results of prescriptions filled utilizing an insurance card, but also of those prescriptions filled with no universal insurance card, including those not utilizing any type of insurance or discount card, referred to as “CASH” customers, discount cards, and so on. This information will then be sent back to the prescriber and/or pharmacy. The prescriber and pharmacist will then be able to evaluate the information and determine which course of action to take.

By running a universal prescription database search for all prescriptions written, filled and dispensed, new prescriptions and refills, prescribers and pharmacists advantageously have access to substantially all relevant prescription information pertaining to the current prescription being filled, regardless of who prescribed the prescription and where other prescriptions were filled. As a result, substantially all prescription information can pass through one universal prescription database, and all prescribers and pharmacists can access the universal prescription database. As shown in FIG. 5, all prescriptions ultimately end up in one column, illustrating that all information has been transmitted to and stored in the universal prescription database to be used for this, and all other relevant future prescriptions.

It will readily be appreciated by those of ordinary skill in the art that if the universal prescription database described above were adopted by all prescribers and pharmacies, and if the universal prescription database were checked and updated for all prescriptions filled and refilled, the advantages and overall health improvements to society would be dramatic. Such a system and method would provide a simple, accurate, inexpensive method for prescribers and pharmacists to check for the same issues that are responsible for today's preventable medication errors (by utilizing a complete medication history (i.e., from multiple sources, including those prescription claims not being processed through a prescription insurance carrier)). In addition, the system and method described herein provides a system with which to track prescribing trends for narcotics as well as narcotic abuse. Finally, the system and method described herein would preferably require every person to have a unique patient identifier in the universal prescription database in order to have a prescription filled, or refilled.

FIGS. 8A-13 show examples of using an embodiment of the present disclosure to achieve a better outcome than is possible with conventional systems. FIG. 8A is a sample screenshot showing displayable information sent as a response returned to a remote terminal from the script server/universal prescription database server illustrating an “EARLY REFILL” problem warning and the selectable options available to the user terminal. FIG. 8B show exemplary screenshots of information displayed in a remote terminal, in response to a user's selection of a “PAYMENT INFORMATION” option of FIG. 8A. FIG. 8C is a sample screen shot showing confirmation of the data stored by the universal prescription database server for reporting purposes when a pharmacist chooses the “PHARMACIST OVERRIDE” option to override the early refill warning of FIG. 8A. In the example of FIGS. 8A-8C, a patient fills a prescription for Percocet 5/325 mg at an independent pharmacy (pharmacy 1). The prescription was written by Doctor “A” for a quantity of 120 tablets with instructions for use that would indicate that the prescription should last no less than 30 days. The patient utilizes a universally accepted insurance card and pays the designated copay.

Three days later, the same patient makes an appointment to be seen by a second physician, Doctor “B.” When asked to complete a new patient questionnaire, he makes no reference to any other physician that he is seeing, particularly Doctor “A.” He proceeds to describe his aliments to Doctor “B” and indicates to Doctor “B” that he has seen the best relief from Percocet 5/325 mg. Doctor “B” in turn prescribes the patient Percocet 5/325 mg, a quantity of 120 tablets, to be take 1 tablet every 6 hours only as needed for pain. This prescription should last no less than 30 days. After leaving the office, the patient proceeds to a retail pharmacy (pharmacy 2). He presents the prescription to the pharmacist, registers as a new patient and indicates that he has no insurance. The pharmacist proceeds to enter the prescription into the pharmacy computer system. A series of checks is performed by the computer to check for drug allergies, negative drug-disease state interactions, negative drug-drug interactions, duplicate therapies, early refills (overuse of a medication), and other potential negative problems. Seeing as the patient had never had any prescriptions filled at this pharmacy or any other within the same chain, no errors are reported. After this initial check, the prescription record is then submitted to the universal prescription database. In real-time, the pharmacist receives an error report instantly indicating an “EARLY REFILL” problem, shown in FIG. 8A. The error report showed that the same patient had the same prescription filled and dispensed three days earlier at an independent pharmacy. As part of the message received from universal prescription database, the pharmacist also was informed that the patient did in fact have insurance (FIG. 8B). As a result, the pharmacist did not fill the prescription, called the prescribing physician to inform him of the other prescription recently filled, and destroyed the prescription per the physician's request (prescriber's request). FIG. 8C shows an override option, which will be discussed more below.

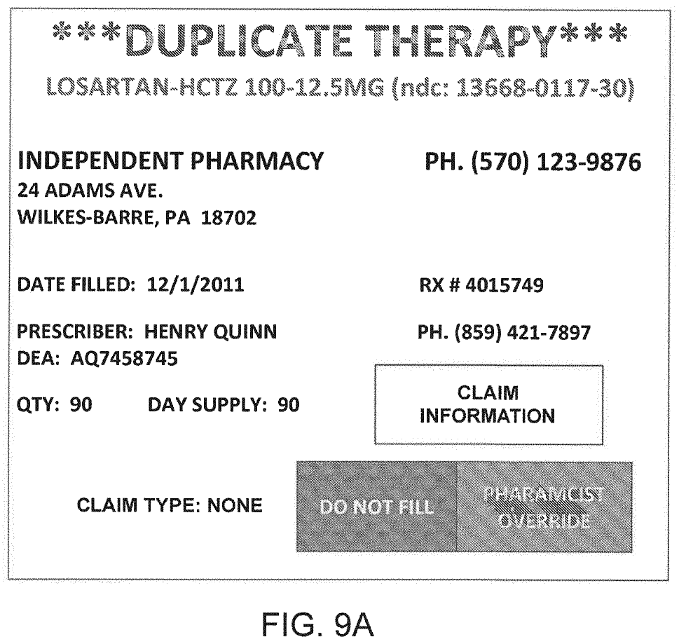

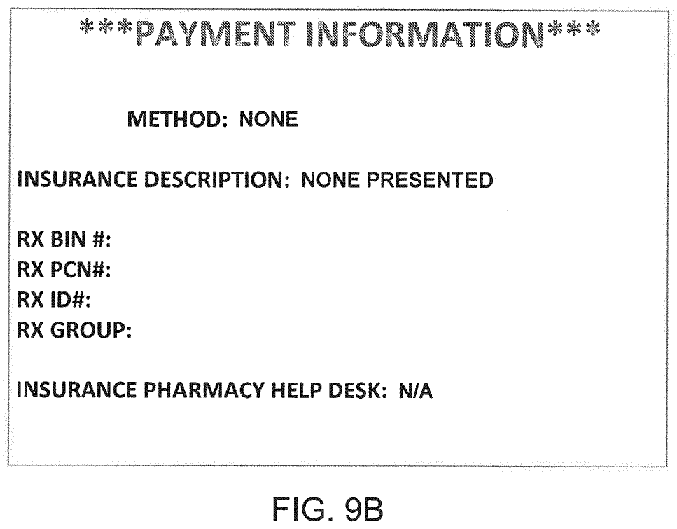

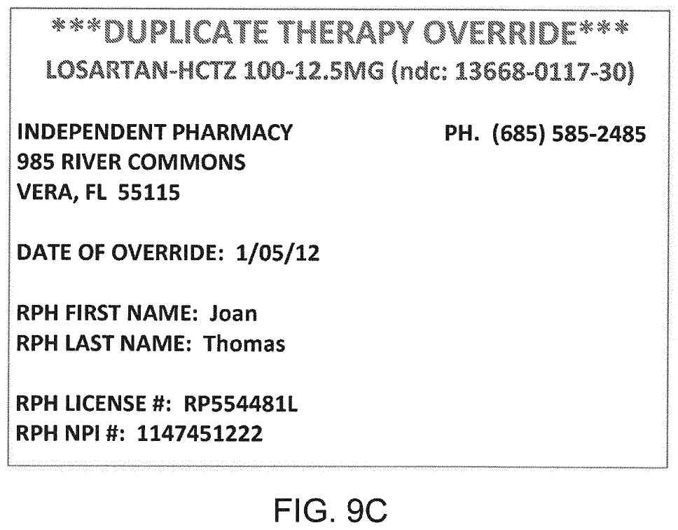

FIG. 9A is a sample screen shot showing displayed information sent as a response returned to a remote terminal from the script server/universal prescription database server illustrating a “DUPLICATE THERAPY” problem warning and the selectable options available to the user terminal. FIG. 9B shows exemplary screenshots of information displayed in a remote terminal, in response to a user's selection of a “PAYMENT INFORMATION” option of FIG. 9A where the patient does not have a universally-accepted insurance card. FIG. 9C is a sample screen shot showing confirmation of the data stored by the universal prescription database server for reporting purposes when a pharmacist chooses the “PHARMACIST OVERRIDE” option to override the “DUPLICATE THERAPY” warning of FIG. 9A.

In a second example, as illustrated in FIGS. 9A-9C, an elderly woman arrives for a week-long visit to see her daughter, who lives a few states away. While packing for the trip, she places in an overnight bag all of her current medications including two medications to control her blood pressure (Hyzaar and Diovan), one medication to control her heart rate (Digoxin) and one medication to prevent blood clots (Warfarin). During her flight, she began experiencing slight chest pains and shortness of breath. So not to alarm anyone, she doesn't tell anyone until the plane lands. Upon landing, an ambulance takes her to the nearest hospital for evaluation. After a few hours, she is seen by an emergency room physician who looks over the list of medications that she provided to them. While she did remember to tell the physician that she was being treated with Hyzaar, Digoxin and Warfarin, she forgot to tell him of the Diovan. After completing all necessary tests, it was determined that the patient had experienced a panic attack. In addition, her blood pressure was a bit higher that designated. The ER physician decided to keep her on all of her current medications but to also add another anti-hypertensive medication to control her blood pressure better. She was advised to continue all previous medication as well as filling the new one immediately and beginning it. Upon leaving the hospital, she proceeds to the nearest pharmacy, a small independent store around the corner. She registers with the pharmacist and informs her that she does not have insurance. The pharmacist proceeds to process the new prescription. As soon as the pharmacist transmits the claim into the universal prescription database, a “DUPLICATE THERAPY problem (FIG. 9A) response (including displayable information) is returned. The pharmacist recognizes that the patient was already on two anti-hypertensive medications. The pharmacist asks the patient if she notified the ER doctor of this and she said that she had not. The pharmacist calls the ER to speak with the prescribing doctor to inform him of this. As a result, the doctor told the pharmacist to cancel the prescription and inform the patient to simply continue taking the medications she was currently on.

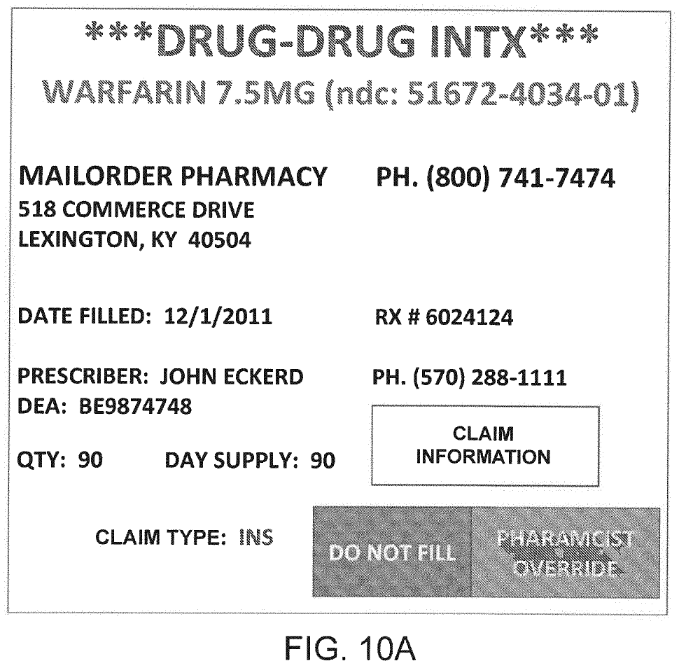

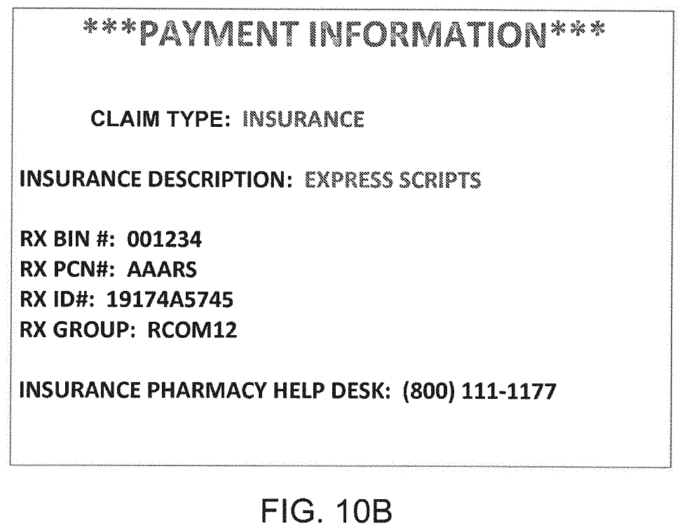

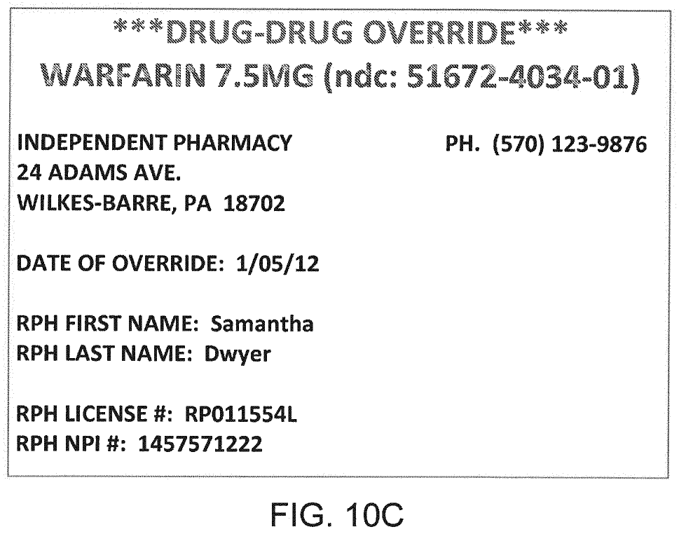

FIG. 10A is a sample screen shot illustrating displayable information sent as a response returned to a remote terminal from the script server/universal prescription database server illustrating an “DRUG-DRUG INTERACTION” (INTX) problem warning and the selectable options available to the user terminal. FIG. 10B shows an exemplary screenshot of information displayed in a remote terminal, in response to a user's selection of a “PAYMENT INFORMATION” option of FIG. 10A. FIG. 10C is a sample screen shot showing confirmation of the data stored by the universal prescription database server for reporting purposes when a pharmacist chooses the “PHARMACIST OVERRIDE” option to override the “DRUG-DRUG INTERACTION” (INTX) warning of FIG. 10A.

In a third example, as shown in FIGS. 10A-10C, an elderly man is living in a high rise housing complex. He is on a fixed income and is very cautious of what he spends. As a result, he gets a variety of maintenance prescriptions filled at 2 separate, unrelated pharmacies. He utilizes his prescription insurance card at one pharmacy (Pharmacy 1) to fill some prescriptions, and pays cash for other prescriptions at another pharmacy (Pharmacy 2). He utilizes the second pharmacy to take advantage of the $3 prescriptions they offer, which is cheaper than utilizing his prescription card. One day complains to his primary physician about a high fever and overall weakness. After a complete examination, the physician determines that the patient has a bacterial infection and prescribes the antibiotic Flagyl to treat it. He hands the prescription to the patient, who then proceeds to the pharmacy. Arriving at Pharmacy 1, the patient presents the prescription to the pharmacist. Upon entering the prescription data into the pharmacy computer system, the pharmacist runs a check on all of the current medications that the patient has filled at this pharmacy. As no problems are reported, the prescription is then sent to the universal prescription database for a more extensive check. Immediately, the pharmacist receives a message indicating a severe “DRUG-DRUG INTERACTION” problem (FIG. 10A) with a prescription that the patient routinely has filled at Pharmacy 2, Warfarin. Immediately, the pharmacist informs the patient of the interaction, contacts the physician for an alternative medication and proceeds to safely fill the new antibiotic.

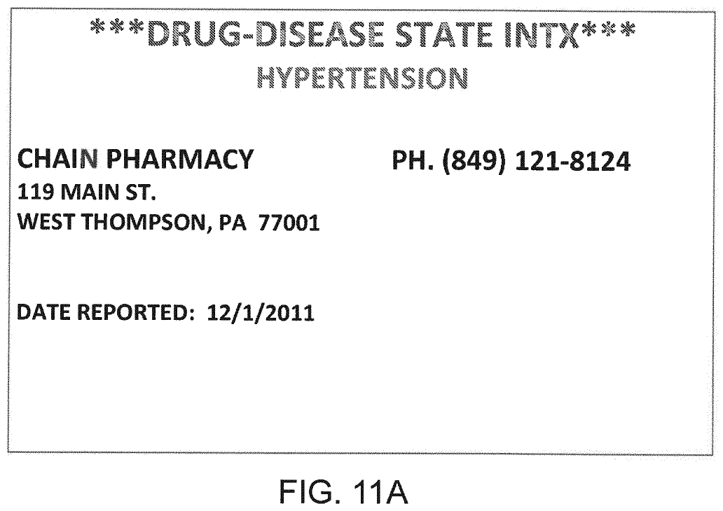

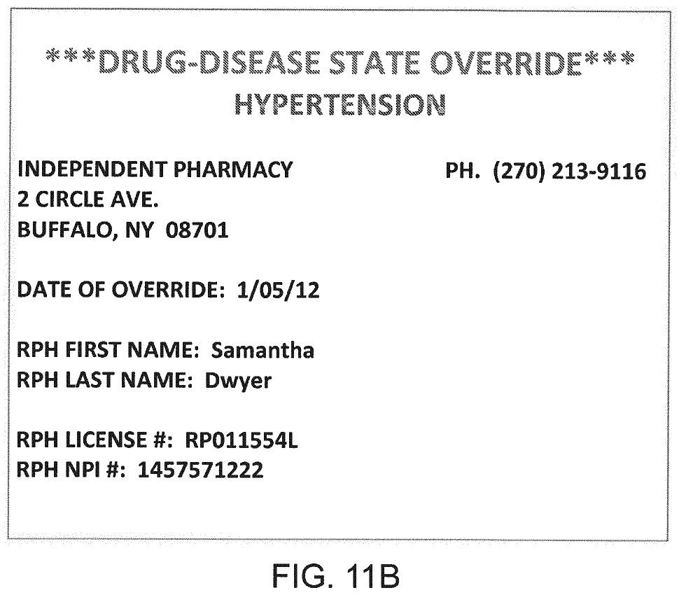

FIG. 11A shows a sample screenshot illustrating information displayed by the remote terminal, which is received in a response returned to the remote terminal from the script server/universal prescription database, the displayed information showing a “DRUG-DISEASE STATE INTERACTION” (INTX) problem warning. FIG. 11B is a sample screen shot showing confirmation of the data stored by the universal prescription database server for reporting purposes when a pharmacist chooses the “PHARMACIST OVERRIDE” option to override the “DRUG-DRUG DISEASE STATE INTERACTION” (INTX) warning of FIG. 11A.

In a second example, as shown in FIGS. 11A-11C, a patient presents to a pharmacy for the first time. Upon registering as a new patient, he informs the pharmacist that the only medical condition that he is being treated for is hypertension (high blood pressure). He presents his prescription drug card, which was issued by the state department of welfare. This particular insurance card is only accepted in the state in which it was issued (in this case, Pennsylvania). He routinely gets his hypertensive medications filled at the same pharmacy. As a result, the universal prescription database has an up-to-date record of his drug allergies and health conditions. While on vacation in upstate New York, he begins to develop flu-like symptoms and decides to visit an urgent care facility. This facility has no record of this patient in their system. As a result, they must rely on the patient giving them an accurate, up-to-date medical history, including drug allergies and health conditions. Being in a hurry to get back to his hotel and rest, he forgets to inform the physician that he is being treated for hypertension. Prior to prescribing anything, the physician utilizes an in-house computer to transmit the prescriptions that he wishes to prescribe the patient to the universal prescription database. In real-time, the prescriber receives notification that the patient is currently taking a hypertensive medication (FIG. 11A), which is a contraindication for the decongestant that he wanted to prescribe. The physician consults the patient, who immediately remembered that he forgot to tell the doctor of this. As a result, the physician was able to switch the medication to something safer.

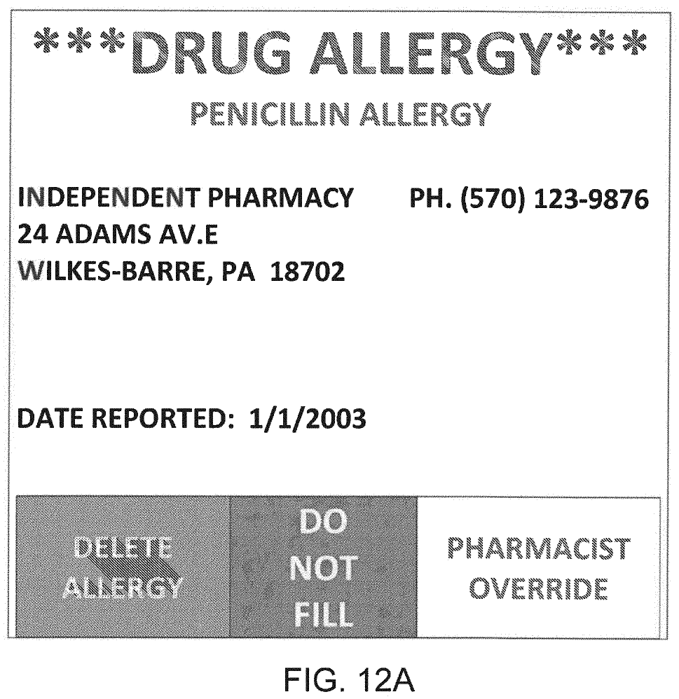

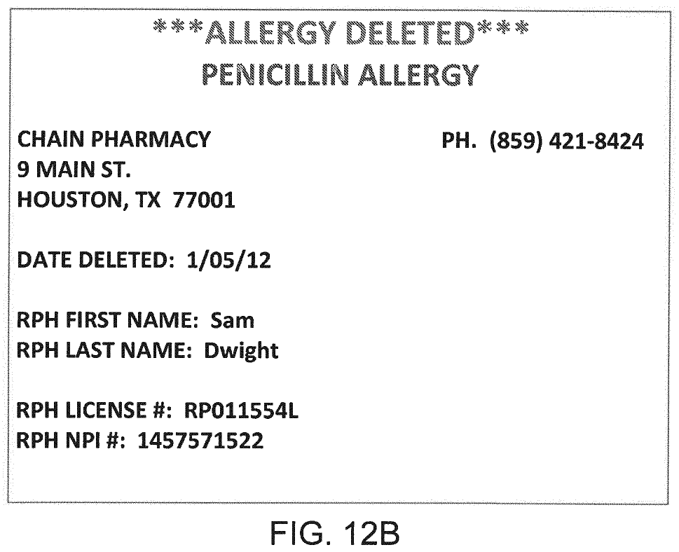

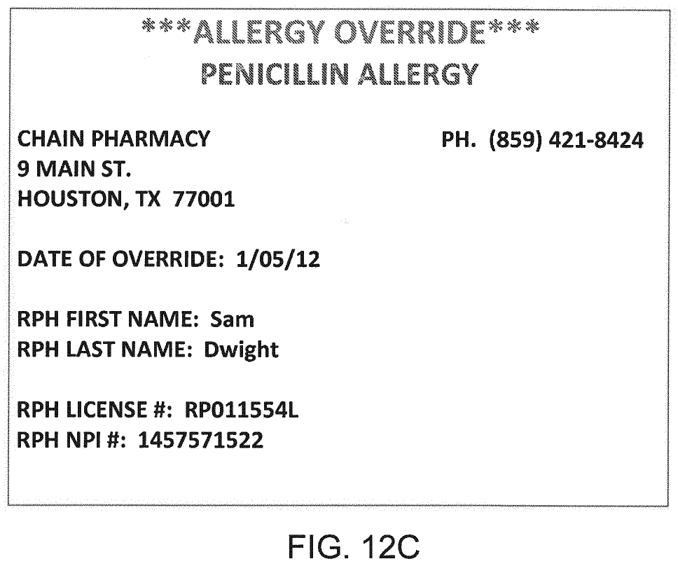

FIG. 12A shows a sample screenshot illustrating information displayed by the remote terminal, which is received in a response returned to the remote terminal from the script server/universal prescription database, the displayed information showing a “DRUG ALLERGY” problem warning and the selectable options available to the user terminal. FIG. 12B is a sample screenshot showing confirmation of the data stored by the universal prescription database server for reporting purposes when a pharmacist chooses the “DELETE ALLERGY” option to override the “DRUG ALLERGY” warning of FIG. 12A. FIG. 12C is a sample screenshot showing confirmation of the data stored by the universal prescription database server for reporting purposes when a pharmacist chooses the “PHARMACIST OVERRIDE” option to override the “DRUG ALLERGY” warning of FIG. 12A.

In a fifth example, as shown by FIGS. 12A-12C, a patient presents to the independent pharmacy (pharmacy 1) that he uses when he needs a prescription filled to inform his pharmacist that he has recently discovered that he has a drug allergy to Penicillin. The pharmacist updates his patient profile to indicate the newly identified drug allergy. Not long after, the patient is prescribed Azithromycin to treat a sore throat. During the filling process, this prescription data is transferred into the universal prescription database to check for any problems, such as drug allergies, negative drug-disease state interactions, negative drug-drug interactions, duplicate therapies, early refills (overuse of a medication), and other potential negative problems. The universal prescription database updates the patient's centralized profile to update the newly reported Penicillin allergy. Years pass and luckily the patient has not had a need for any prescriptions to be filled. During that time, however, the patient moved to a new state and was forced to find a new family care physician after coming down with an illness. During his first visit to the new physician, he fills out a new patient form. While filling out the form, he forgets to indicate the drug allergy to Penicillin that his previous physician identified years back. As the present physician was unaware of the allergy, he prescribes Penicillin to treat his condition. The patient then proceeds to a chain pharmacy (pharmacy 2) to have the prescription filled. As he has never used this particular pharmacy before, or any within the same chain, he takes a moment to provide his information to the pharmacist to enter in the computer system. Again forgetting his allergy to Penicillin, he neglects to inform the pharmacist of his drug allergy. The pharmacist proceeds to fill the prescription. After entering the prescription data into the pharmacy system, no problems are identified. Proceeding next to the step, the prescription data is then sent to the universal prescription database. After running a complete, detailed check an error is reported back to the pharmacist (FIG. 12A) indicating that the patient has in the past reported an allergy to Penicillin. The result identified the date the allergy was reported as well as which pharmacy reported it. The pharmacist asks the patient about the reported allergy and confirmed that it was a true allergy. As a result, the pharmacist chose not to fill the prescription and called the prescribing physician for an alternative.

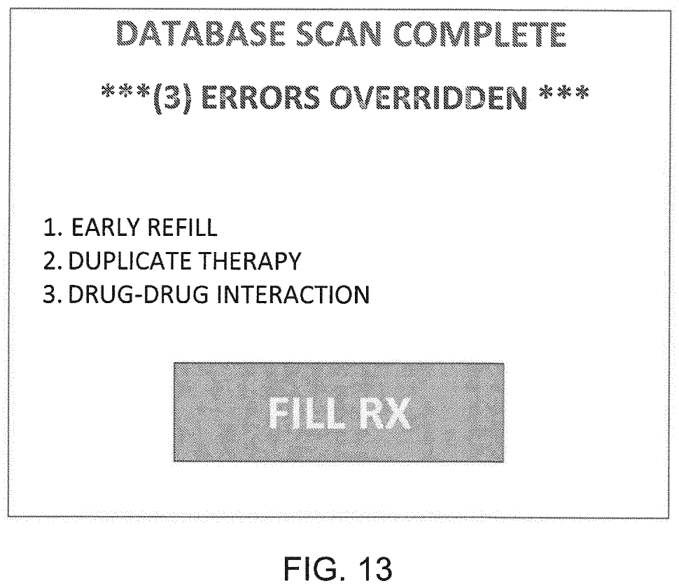

FIG. 13 is a sample screenshot illustrating the response returned to a remote from the universal prescription database indicating that a complete search was performed, how many errors were overridden, and what those error were. In a sixth example, as illustrated by FIG. 13, a local Drug Enforcement Agency (DEA) agent presents to number of local pharmacy's inquiring about the prescribing habits of a local physician who has come under investigation for over-prescribing narcotics. After an extensive investigation, it was determined that an extraordinary number of prescriptions were prescribed by the physician over the period of 2 years. As part of the investigation, the DEA, through the appropriate legal steps, also requested records from the universal prescription database to support the over-prescribing habits. In the report, all of the dispensed prescriptions written by this physician were supplied. The report showed the following for each dispensed prescription: the date the prescription was written, the date the prescription was dispensed, the prescribing physician, the prescribing physician's DEA number, the prescribing physician's NPI number, the prescribing physician's state license number(s), the prescribing physician's full office address, the prescribing physician's office phone and facsimile numbers, the name of the drug, the drug strength, the quantity dispensed, and the day supply of the prescription. In addition, the report also showed for each dispensed prescription the name of the pharmacy that it was dispensed at, the full address of the pharmacy, the phone number of the pharmacy, the dispensing pharmacist's full name, and the dispensing pharmacist's NPI number and state license number. Finally, and most importantly, the report showed that for each prescription that was filled, the universal prescription database reported numerous errors that were reported back to the pharmacist for review. Each time the pharmacist overrode an error, the universal prescription database recorded the type of error(s), the name of the pharmacist who overrode the error(s), the overriding pharmacist's NPI number and state license number(s). Upon review of the report, it was identified that the majority of the prescriptions prescribed by the physician were filled at the same pharmacy and dispensed by the same pharmacist. By means of evaluating the recorded overrides of this specific pharmacist, it was determined that the prescriber and physician were working together.

In each of the above examples, it can be seen that the use of a universally-utilizable prescription database advantageously provides prescribers and pharmacists with important information that they can use to achieve better outcomes for patients, including avoiding negative drug interactions, and preventing abuse of prescription drugs. In each of the examples, conventional systems are insufficient to provide the pharmacist with sufficient information to achieve these better outcomes. It will also be appreciated that the more prescribers and pharmacies that participate in the universal prescription database, the more effective it will be.

The below chart compares preferred features of an exemplary embodiment of the present disclosure to a conventional system and database maintained by, for example, an insurance company. While the disclosure has been shown and described with reference to certain embodiments thereof, it will be understood by those skilled in the art that various changes in form and details may be made therein without departing from the spirit and scope of the disclosure as defined by the appended claims.

| | | Universal | |
|---|---|---|---|
| | | Prescription | Conven- |
| | | Database | tional |
| General Information | | System | Systems |
| 1. | Uses a centralized computer database | X | X |
| 2. | Database communicates centrally with | X | X |
| | all pharmacies nationwide | | |
| 3. | Database could be utilized by | X | X |
| | pharmacists | | |
| 4. | Database requires a separate log in by | | |
| | pharmacist to utilize the database | | |
| 5. | Database could be accessed by | X | |
| | prescribers | | |
| 6. | Database requires a separate log-in by | X | |
| | prescriber to utilize the database | | |
| 7. | Database tracks ALL prescriptions | X | |
| | filled and dispensed | | |
| 8. | Database tracks filling history for ALL | X | |
| | medications for each individual | | |
| 9. | Database tracks prescribing habits of | X | X |
| | prescriber for ALL medications | | |
| 10. | Database will securely store and | X | |
| | transmit ONLY relevant data with | | |
| | regard to ALL prescriptions attempting | | |
| | to be filled | | |
| 11. | Database will provide responses in | X | X |
| | real-time to remote terminals | | |
| 12. | Database has reporting capabilities | X | X |

| | | Universal | |
|---|---|---|---|
| | | Prescription | Conven- |
| | | Database | tional |
| Use of System | | System | Systems |
| 1. | System could be utilized by: | | |
| | Pharmacists | X | X |
| | Prescribers | X | X |
| | Hospital Staff (other than prescribers) | | |
| | Insurance Companies | X | X |
| | Government Agencies | X | |
| 2. | Use of the system is initiated at the | | |
| | level of: | | |
| | Pharmacist | X | X |
| | Prescriber | X | |
| 3. | Database will “communicate” with | X | |
| | prescriber/pharmacist by sending relevant | | |
| | information back to the remote location | | |
| | via secure messaging system | | |
| 4. | Database will store data immediately (in | X | X |
| | real-time) for immediate use by multiple | | |
| | remote locations | | |
| 5. | The decision to prescribe, fill and dispense | X | |
| | a prescription is based on the information | | |
| | sent back to the prescriber and pharmacist | | |
| | (at remote locations) from the universal | | |
| | database | | |

| | | Universal | |
|---|---|---|---|
| | | Prescription | Conven- |
| | | Database | tional |
| Information | | System | Systems |
| 1. | Data required for the system to work: | | |
| | Patient's full name | X | X |
| | Patient's date of birth | X | X |
| | Patient's social security number | X | |
| | Drug name | X | X |
| | Drug strength | X | X |
| | Drug quantity | X | X |
| | Drug sig. (instructions for use) | X | X |
| | Day supply of prescription | X | X |
| | Prescriber's full name | X | X |
| | Prescriber's DEA # | X | X |
| | Prescriber's NPI # | X | X |
| | Prescriber's state license # | X | X |
| | Prescriber's office address | X | |
| | Prescriber's office phone # | X | |
| 2. | After filling each prescription, both new | | |
| | and refills, the system will store: | | |
| | Patient's full name | X | X |
| | Patient's full address | X | |
| | Patient's phone # | X | |
| | Patient's date of birth | X | X |
| | Patient's social security number | X | |
| | Prescriber's full name | X | X |
| | Prescriber's full office address | X | |
| | Prescriber's office phone # | X | |
| | Prescriber's office facsimile # | X | |
| | Prescriber's DEA # | X | X |
| | Prescriber's NPI # | X | X |
| | Prescriber's state license # | X | X |
| | Date the prescription was written | X | X |
| | Date the prescription was dispensed | X | X |
| | Drug name | X | X |
| | Drug NDC # | X | X |
| | Drug strength | X | X |
| | Drug quantity | X | X |
| | Drug sig. (instructions for use) | X | X |
| | Day supply of prescription | X | X |
| | Pharmacy name | X | X |
| | Pharmacy's full address | X | |
| | Pharmacy's phone # | X | |
| | Dispensing pharmacist's full name | X | |
| | Dispensing pharmacist's state license # | X | |
| | Dispensing pharmacist's NPI # | X | |
| | Payment information | X | |
| | If insurance used, the following is | | |
| | obtained: | | |
| | 1. BIN # | X | |
| | 2. PCN # | X | |
| | 3. RxID # | X | |
| | 4. Rx Group # | X | |
| | 5. Person Code | X | |
| | 6. Insurance pharmacy help desk phone # | X | |
| 3. | Each prescription run through the universal | | |
| | prescription database will return the | | |
| | following information relevant to each | | |
| | prescription attempting to be filled: | | |
| | Patient's full name | X | X |
| | Patient's full address | X | |
| | Patient's phone # | X | |
| | Patient's date of birth | X | X |
| | Patient's social security number | X | |
| | Prescriber's full name | X | X |
| | Prescriber's full office address | X | |
| | Prescriber's office phone # | X | |
| | Prescriber's office facsimile # | X | |
| | Prescriber's DEA # | X | X |
| | Prescriber's NPI # | X | X |
| | Prescriber's state license # | X | X |
| | Date the prescription was written | X | X |
| | Date the prescription was dispensed | X | X |
| | Drug name | X | X |
| | Drug NDC # | X | X |
| | Drug strength | X | X |
| | Drug quantity | X | X |
| | Drug sig. (instructions for use) | X | X |
| | Day supply of prescription | X | X |
| | Pharmacy name | X | X |
| | Pharmacy's full address | X | |
| | Pharmacy's phone # | X | |
| | Filling pharmacist's full name | X | |
| | Filling pharmacist's state license # | X | |
| | Filling pharmacist's NPI # | X | |
| | Payment information | X | |
| | If insurance used, the following is | | |
| | obtained: | | |
| | 1. BIN # | X | |
| | 2. PCN # | X | |
| | 3. RxID # | X | |
| | 4. Rx Group # | X | |
| | 5. Person Code | X | |
| | 6. Insurance pharmacy help desk phone # | X | |
| 4. | The universal prescription database will | X | |
| | store all information for every prescription | | |
| | filled (new or refill) by all persons regard- | | |
| | less of whether or not the person utilized | | |
| | a universally accepted insurance card. | | |
| 5. | Once verified through the universal | X | |
| | prescription database, the database will | | |
| | then allow for claim transmission to all | | |
| | insurance company computer systems | | |
| | (if patient is utilizing an insurance card) | | |
| | to verify the insurance company's | | |
| | restrictions/limitations | | |

| | | Universal | |
|---|---|---|---|
| | | Prescription | Conven- |
| | | Database | tional |
| Reporting Capabilities | | System | Systems |
| 1. | System tracks prescribing habit of | X | |
| | prescriber for ALL prescriptions written | | |
| 2. | Prescriber prescribing report includes: | | |
| | Prescriber's full name | X | |
| | Prescriber's full office address | X | |
| | Prescriber's office phone # | X | |
| | Prescriber's office facsimile # | X | |
| | Prescriber's DEA # | X | |
| | Prescriber's NPI # | X | |
| | Prescriber's state license # | X | |
| | Patient's full name | X | |
| | Patient's full address | X | |
| | Patient's phone # | X | |
| | Patient's date of birth | X | |
| | Patient's social security number | X | |
| | Date the prescription was written | X | |
| | Date the prescription was dispensed | X | |
| | Drug name | X | |
| | Drug NDC # | X | |
| | Drug strength | X | |
| | Drug quantity | X | |
| | Drug sig. (instructions for use) | X | |
| | Day supply of prescription | X | |
| | Pharmacy name | X | |
| | Pharmacy's full address | X | |
| | Pharmacy's phone # | X | |
| | Filling pharmacist's full name | X | |
| | Filling pharmacist's state license # | X | |
| | Filling pharmacist's NPI # | X | |
| | Payment information | X | |
| | If insurance used, the following is | | |
| | obtained: | | |
| | 1. BIN # | X | |
| | 2. PCN # | X | |
| | 3. RxID # | X | |
| | 4. Rx Group # | X | |
| | 5. Person Code | X | |
| | 6. Insurance pharmacy help desk phone # | X | |
| 2. | System tracks pharmacist over ride | | |
| | history for ALL prescriptions written | | |
| 3 | Pharmacist over ride report includes: | | |
| | The type of override (e.g. early refill) | X | |
| | Filling pharmacist's full name | X | |
| | Filling pharmacist's state license # | X | |
| | Filling pharmacist's NPI # | | |
| | Prescriber's full name | X | |
| | Prescriber's full office address | X | |
| | Prescriber's office phone # | X | |
| | Prescriber's office facsimile # | X | |
| | Prescriber's DEA # | X | |
| | Prescriber's NPI # | X | |
| | Prescriber's state license # | X | |
| | Patient's full name | X | |
| | Patient's full address | X | |
| | Patient's phone # | X | |
| | Patient's date of birth | X | |
| | Patient's social security number | X | |
| | Date the prescription was written | X | |
| | Date the prescription was dispensed | X | |
| | Drug name | X | |
| | Drug NDC # | X | |
| | Drug strength | X | |
| | Drug quantity | X | |
| | Drug sig. (instructions for use) | X | |
| | Day supply of prescription | X | |
| | Pharmacy name | X | |
| | Pharmacy's full address | X | |
| | Pharmacy's phone # | X | |
| | Payment information | X | |
| | If insurance used, the following is | | |
| | obtained: | | |
| | 1. BIN # | X | |
| | 2. PCN # | X | |
| | 3. RxID # | X | |
| | 4. Rx Group # | X | |
| | 5. Person Code | X | |
| | 6. Insurance pharmacy help desk phone # | X | |
| 3. | System tracks patient fill history of ALL | | |
| | medications dispensed | | |
| 4 | Patient fill history report includes: | | |
| | Patient's full name | X | X |
| | Patient's full address | X | |
| | Patient's phone # | X | |
| | Patient's date of birth | X | X |
| | Patient's social security number | X | |
| | Prescriber's full name | X | X |
| | Prescriber's full office address | X | |
| | Prescriber's office phone # | X | |
| | Prescriber's office facsimile # | X | |
| | Prescriber's DEA # | X | X |
| | Prescriber's NPI # | X | X |
| | Prescriber's state license # | X | X |
| | Date the prescription was written | X | X |
| | Date the prescription was dispensed | X | X |
| | Drug name | X | X |
| | Drug NDC # | X | X |
| | Drug strength | X | X |
| | Drug quantity | X | X |
| | Drug sig. (instructions for use) | X | X |
| | Day supply of prescription | X | X |
| | Pharmacy name | X | X |
| | Pharmacy's full address | X | |
| | Pharmacy's phone # | X | |
| | Filling pharmacist's full name | X | |
| | Filling pharmacist's state license # | X | |
| | Filling pharmacist's NPI # | X | |
| | Payment information | X | |
| | If insurance used, the following is | | |
| | obtained: | | |
| | 1. BIN # | X | |
| | 2. PCN # | X | |
| | 3. RxID # | X | |
| | 4. Rx Group # | X | |
| | 5. Person Code | X | |
| | 6. Insurance pharmacy help desk phone # | X | |

1. A system for checking for problems with filling a new prescription drug request or a refill of a previously-filled prescription drug request, the system comprising:
a remote client terminal comprising:
a display;
a network communications interface configured to communicate with a universal prescription database server over a computer network;
a memory configured to store prescription drug records in a local database, each prescription drug record being stored in association with at least one unique patient identifier; and
a processor programmed to:
(1) receive, based on user input, a prescription authorization request including an associated unique patient identifier of a patient, a drug identifier, a prescriber identifier, and either a unique identifier of an insurance company or an indication that the patient does not have insurance;
(2) determine, based on a check of the local database, whether one or more drug-related filling problems exist with the received prescription authorization request;
(3) if at least one drug-related filling problems exists with the check of the local database, output to the display visibly-perceptible alert notification information identifying at least one of the one or more problems determined to exist; and
(4) if no drug-related filling problems exist with the check of the local database, and the remote client terminal is associated with a retail chain database server:
(A) transmit, to the retail chain database server, the prescription authorization request including the associated unique patient identifier and the drug identifier;
(B) receive, from the retail pharmacy chain database server, a retail chain indication of whether any drug-related filling problems exist with the prescription authorization request; and
(C) if the received retail chain indication indicates that at least one drug-related filling problems exists, output to the display the visibly-perceptible alert notification information identifying at least one of the one or more problems determined to exist; and
(5) if no drug-related filling problems exist with the check of the local database and the retail indication indicates that no drug-related filling problems exist with the prescription authorization request or the remote client terminal is not associated with a retail chain database server:
(A) transmit, to the universal prescription database server, the prescription authorization request including the associated unique patient identifier, the drug identifier and either the unique identifier of the insurance company or the indication that the patient does not have insurance;
(B) receive, from the universal prescription database server, a universal indication of whether any drug-related filling problems exist with the prescription authorization request; and
(C) if the received universal indication indicates that at least one drug-related filling problems exists, output to the display the visibly-perceptible alert notification information identifying at least one of the one or more problems determined to exist;
(6) if the received universal indication indicates that no drug-related problems exist:
(A) if the received prescription authorization request includes the indication that the patient does not have insurance, output to the display information indicating that no drug-related problems exist with filling/dispensing the prescription authorization request;
(B) if the received prescription authorization request includes the unique identifier of the insurance company:
(i) transmit, to a prescription database server of an insurance company, based on the unique identifier of the insurance company, the prescription authorization request including the associated unique patient identifier and the drug identifier;
(ii) receive, from the prescription database server of the insurance company, an insurance indication of whether one or more drug-related filling problems exist with the prescription authorization request; and
(iii) if the received insurance indication indicates that at least one drug-related filling problems exists, output to the display the visibly-perceptible alert notification information identifying at least one of the one or more problems determined to exist; and
(C) if the received insurance indication indicates that no drug-related filling problems exist with the prescription authorization request output to the display information indicating that no drug-related problems exist with filling/dispensing the prescription authorization request; and
(7) upon entry of user input indicating that the prescription drug request has been filled and dispensed, transmit, to the universal prescription database server, an indication of the filling and dispensing of the prescription drug request and additional information including the fill date, and the prescriber identifier; and
a universal prescription database server comprising:
a memory that stores a universal prescription database;
a network communications interface configured to communicate with the remote client terminal over the computer network; and
a processor programmed to:
(1) receive the prescription authorization request, over the computer network via the network communications interface from the remote client terminal, the prescription authorization request including the associated unique patient identifier, the drug identifier, and either the unique identifier of the insurance company or the indication that the patient does not have insurance;
(2) compare the drug identifier in the received prescription authorization request with one or more drug identifiers in existing prescription records associated with the same unique patient identifier stored in the universal prescription database;
(3) if, based on the results of the comparison with the universal database, at least one drug-related filling problem exists, transmit information identifying at least one of the one or more drug-related filling problems determined to exist;
(4) if, based on the results of the comparison with the universal prescription database, no drug-related filling problems exist with the received prescription authorization request, transmit information indicating that no drug-related problems exist with filling the prescription authorization request; and
(5) upon receipt, from the remote terminal, of the indication that the requested prescription has been filled and dispensed and the additional information, update the universal prescription database to indicate that the requested prescription was filled and dispensed with the associated additional information.

2. The system of claim 1, wherein
the memory is further configured to store prescriber records, pharmacy records, and dispenser's records in the universal prescription database,
the at least one unique patient identifier comprises one of: (i) a set of three parameters consisting of: a patient first name, a patient last name, and a patient date of birth, and (ii) a patient social security number,
the prescription records comprise a National Drug Code number (NDC#) or other unique drug identifier, a drug strength, a dispense quantity, a day supply, instructions for use, a prescription written date, prescriber identification information, and dispenser identifiers,
the prescriber identification information comprises one or more of a prescriber's first name, a prescriber's last name, a prescriber's DEA number, a prescriber's NPI number, a prescriber's state license number(s), a prescriber's office phone number, a prescriber's address, and a prescriber's facsimile number, and
the dispenser identification information comprise one or more of a pharmacy name, a pharmacy number, a pharmacy address, a pharmacy phone number, a pharmacy facsimile number, a dispensing pharmacist first name, a dispensing pharmacist last name, a dispensing pharmacist state license number, and a dispensing pharmacist NPI number.

3. The system of claim 1, wherein the network communications interface is a secure communications interface.

4. The system of claim 1, wherein the drug-related problems include at least one of: a drug allergy, a negative drug-disease state interaction, a negative drug-drug interaction, a duplicate therapy, an early refill, and an overuse of a medication.

5. The system of claim 4, wherein the processor is further programmed to:
assign a rating to each of the drug-related problems based on a severity of the problem; and
include the rating in the transmitted information identifying the at least one of the one or more drug-related filling problems determined to exist.

6. The system of claim 1, wherein
the prescription records comprise:
a first prescription record associated with a first unique patient identifier, the first prescription record comprising an insurance company identifier of a first insurance company, and
a second prescription record associated with the first unique patient identifier comprising an insurance company identifier of a second insurance company that is different than the first insurance company.

7. The system of claim 1, wherein each of the prescription records further comprise an insurance field that identifies whether an insurance claim is associated with the prescription record.

8. The system of claim 7, wherein if the insurance field does not indicate that an insurance claim is associated with the prescription record, the insurance field indicates that the prescription was paid for with cash.

9. A method of checking for problems with filling a new prescription drug request or a refill of a previously-filled prescription drug request, the method comprising:
(1) receiving, from a client terminal, based on user input, a prescription authorization request including an associated unique patient identifier of a patient, a drug identifier, a prescriber identifier, and either a unique identifier of an insurance company or an indication that the patient does not have insurance;
(2) determining, based on a check of a local database of the client terminal, whether one or more drug-related filling problems exist with the received prescription authorization request;
(3) if at least one drug-related filling problems exists with the check of the local database, outputting, to a display of the client terminal, visibly-perceptible alert notification information identifying at least one of the one or more drug-related filling problems determined to exist;
(4) if no drug-related filling problems exist with the check of the local database and the remote client terminal is associated with a retail chain database server:
(A) transmitting, over a computer network via a network communications interface from the client terminal to the retail chain database server, the prescription authorization request including the associated unique patient identifier and the drug identifier;
(B) receiving, by the client terminal from the retail chain database server, a retail indication of whether any drug-related filling problems exist with the prescription authorization request; and
(C) if the received retail chain indication indicates that at least one drug-related filling problems exists, outputting, to the display of the client terminal, visibly-perceptible alert notification information identifying at least one of the one or more drug-related filling problems determined to exist; and
(5) if no drug-related filling problems exist with the check of the local database, and either: (i) the retail chain indication indicates that no drug-related filling problems exist with the prescription authorization request, or (ii) the remote client terminal is not associated with a retail chain database server:
(A) transmitting, over the computer network from the client terminal to a universal database server, the prescription authorization request including the associated unique patient identifier;
(B) comparing, by the universal database server, the drug identifier in the received prescription authorization request with one or more drug identifiers in existing prescription records stored in the universal prescription database that are associated with the same unique patient identifier;
(C) if, based on the results of the comparison by the universal prescription database server, at least one drug-related filling problem exists: (i) transmitting, from the universal prescription database server to the client terminal, information identifying at least one of the one or more drug-related filling problems determined to exist, and (ii) outputting to the display of the client terminal visibly-perceptible alert notification information identifying at least one of the one or more drug-related filling problems determined to exist; and
(D) if, based on the results of the comparison of the universal prescription database server, no drug-related filling problems exist with the received prescription authorization request:
(i) if the received prescription authorization request includes the unique identifier of the insurance company:
(a) transmitting, to a prescription database server of the insurance company by the universal database server, based on the unique identifier of the insurance company, the prescription authorization request including the associated unique patient identifier and the drug identifier,
(b) receiving, from the prescription database server of the insurance company by the universal prescription database server, an insurance indication of whether one or more drug-related filling problems exist with the prescription authorization request; and
(c) if the received insurance indication indicates that at least one drug-related filling problems exists, transmitting, by the universal prescription database server to the client terminal, information identifying at least one of the one or more drug-related filling problems determined to exist; and
(ii) if the insurance indication indicates that no drug-related filling problems exist with the prescription authorization request, or if the received prescription authorization request includes the indication that the patient does not have insurance: (a) outputting, to the display by the client terminal, information indicating that no drug-related problems exist with filling the prescription authorization request, (b) receiving, by the client terminal based on user input, information indicating that the requested prescription has been filled and dispensed, (c) transmitting, to the universal prescription database server from the client terminal, information indicating that the requested prescription has been filled and dispensed and additional information including the fill date, and the prescriber identifier, and (d) upon receipt, by the universal prescription database server, of the indication that the requested prescription has been filled and dispensed and the additional information, updating, by the universal prescription database server, the universal prescription database to indicate that the requested prescription was filled and dispensed and storing the associated additional information therewith.

10. The method of claim 9, further comprising storing prescriber records, pharmacy records, and dispenser's records in the universal prescription database of the memory, wherein
the unique patient identifier comprises one or more of: a patient first name, a patient last name, a patient date of birth, and a patient social security number;
the prescription records comprise a National Drug Code number (NDC#), a drug strength, a dispense quantity, a day supply, instructions for use, a prescription written date, prescriber identifiers, and dispenser identifiers,
the prescriber identifiers comprise a prescriber's first name, a prescriber's last name, a prescriber's DEA number, a prescriber's NPI number, a prescriber's state license number(s), a prescriber's office phone number, a prescriber's address, and a prescriber's facsimile number; and
the dispenser identifiers comprise a pharmacy name, a pharmacy number, a pharmacy address, a pharmacy phone number, a pharmacy facsimile number, a dispensing pharmacist first and last name, a dispensing pharmacist state license number, and a dispensing pharmacist NPI number.

11. The method of claim 9, wherein the network communications interface is a secure communications interface.

12. The method of claim 9, wherein the drug-related problems include at least one of: a drug allergy, a negative drug-disease state interaction, a negative drug-drug interaction, a duplicate therapy, an early refill, and an overuse of a medication.

13. The method of claim 12, further comprising assigning a rating to each of the drug-related problems based on a severity of the problem, wherein the rating is included in the transmitted response.

14. The method of claim 9, wherein the prescription records comprise:
a first prescription record associated with a first patient comprising an insurance company identifier of a first insurance company, and
a second prescription record associated with a first patient comprising an insurance company identifier of a second insurance company that is different than the first insurance company.

15. The method of claim 9, wherein each of the prescription records further comprise an insurance field that identifies whether an insurance claim is associated with the prescription record.

16. The method of claim 15, wherein if the insurance field does not indicate that an insurance claim is associated with the prescription record, the insurance field indicates that the prescription was paid for with cash.

17. A system for checking for problems with filling a new prescription drug request or a refill of a previously-filled prescription drug request, the system comprising:
a remote client terminal comprising:
a display;
a network communications interface configured to communicate with a universal prescription database server over a computer network;
a memory configured to store prescription drug records in a local database, each prescription drug record being stored in association with at least one unique patient identifier; and
a processor programmed to:
(1) receive, based on user input, a prescription authorization request including an associated unique patient identifier of a patient, a drug identifier, a prescriber identifier, and either a unique identifier of an insurance company or an indication that the patient does not have insurance;
(2) determine, based on a check of the local database, whether one or more drug-related filling problems exist with the received prescription authorization request;
(3) if at least one drug-related filling problems exists with the check of the local database, output to the display visibly-perceptible alert notification information identifying at least one of the one or more problems determined to exist;
(4) if no drug-related filling problems exist with the check of the local database, and the remote client terminal is associated with a retail chain database server:
(A) transmit, to the retail chain database server, the prescription authorization request including the associated unique patient identifier and the drug identifier;
(B) receive, from the retail pharmacy chain database server, a retail chain indication of whether any drug-related filling problems exist with the prescription authorization request; and
(C) if the received retail chain indication indicates that at least one drug-related filling problems exists: (i) output, to the display, visibly-perceptible alert notification information identifying at least one of the one or more problems determined to exist and an override button, and (ii) when the user indicates, via clicking/pressing the override button, that the prescription was overridden so as to be filled and dispensed, transmit an indication of the overriding and dispensing to the universal prescription database server;
(5) if no drug-related filling problems exist with the check of the local database and the retail indication indicates that no drug-related filling problems exist with the prescription authorization request or the remote client terminal is not associated with a retail chain database server:
(A) transmit, to the universal prescription database server, the prescription authorization request including the associated unique patient identifier, the drug identifier and either the unique identifier of the insurance company or the indication that the patient does not have insurance;
(B) receive, from the universal prescription database server, a universal indication of whether any drug-related filling problems exist with the prescription authorization request; and
(C) if the received universal indication indicates that at least one drug-related filling problems exists: (i) output, to the display, visibly-perceptible alert notification information identifying at least one of the one or more problems determined to exist and an override button, and (ii) when the user indicates, via clicking/pressing the override button, that the prescription was overridden so as to be filled and dispensed, transmit an indication of the overriding and the dispensing to the universal prescription database server;
(6) if the received universal indication indicates that no drug-related problems exist:
(A) if the received prescription authorization request includes the indication that the patient does not have insurance, output to the display information indicating that no drug-related problems exist with filling/dispensing the prescription authorization request;
(B) if the received prescription authorization request includes the unique identifier of the insurance company:
(i) transmit, to a prescription database server of the insurance company, based on the unique identifier of the insurance company, the prescription authorization request including the associated unique patient identifier and the drug identifier;
(ii) receive, from the prescription database server of the insurance company, an insurance indication of whether one or more drug-related filling problems exist with the prescription authorization request; and
(iii) if the received insurance indication indicates that at least one drug-related filling problems exists: (a) output, to the display, visibly-perceptible alert notification information identifying at least one of the one or more problems determined to exist and an override button, and (b) when the user indicates, via clicking/pressing the override button, that the prescription was overridden so as to be filled and dispensed, transmit an indication of the overriding and dispensing to the universal prescription database server; and
(C) if the received insurance indication indicates that no drug-related filling problems exist with the prescription authorization request, output, to the display, information indicating that no drug-related problems exist with filling/dispensing the prescription authorization request; and
(7) upon entry of user input indicating that the prescription drug request has been filled and dispensed, transmit, to the universal prescription database server, an indication of the filling and dispensing of the prescription drug request and additional information including the fill date, and the prescriber identifier; and
a universal prescription database server comprising:
a memory that stores a universal prescription database;
a network communications interface configured to communicate with the remote client terminal over the computer network; and
a processor programmed to:
(1) receive the prescription authorization request, over the computer network via the network communications interface from the remote client terminal, the prescription authorization request including the associated unique patient identifier, the drug identifier, and either the unique identifier of the insurance company or the indication that the patient does not have insurance;
(2) compare the drug identifier in the received prescription authorization request with one or more drug identifiers in existing prescription records associated with the same unique patient identifier stored in the universal prescription database;
(3) if, based on the results of the comparison with the universal database, at least one drug-related filling problem exists, transmit information identifying at least one of the one or more drug-related filling problems determined to exist;
(4) if, based on the results of the comparison with the universal prescription database, no drug-related filling problems exist with the received prescription authorization request, transmit information indicating that no drug-related problems exist with filling the prescription authorization request; and
(5) upon receipt, from the remote terminal, of the indication that the requested prescription has been filled and dispensed and the additional information, update the universal prescription database to indicate that the requested prescription was filled and dispensed with the associated additional information, wherein if the indication indicates that the prescription authorization request was overridden so as to be filled and dispensed, store an indication indicating the prescription authorization request was overridden and filled and dispensed.

18. The system of claim 17, wherein the processor is further programmed to:
capture auditable information regarding the prescriber including a prescriber identification number, when the prescription has been filled or overridden and filled; and
generate displayable report information including prescribing habits of the prescriber based on actually filled prescriptions.

19. The system of claim 17, wherein the processor is further programmed to:
capture auditable information regarding the prescriber including a prescriber identification number, when the prescription has been filled or overridden and filled; and
generate displayable report information including any unordinary amount of prescriptions of controlled substances.

20. The system of claim 17, wherein the processor is further programmed to:
capture or record auditable information regarding the pharmacist/pharmacy including a pharmacist identification number, and/or a pharmacist name, and information regarding the prescriber including a prescriber identification number, when the prescription has been overridden and filled;
determine whether a threshold percentage of prescriptions prescribed by a prescriber were filled at the same pharmacy and dispensed by the same pharmacist;
determine, based on the captured or recorded overrides, a working relationship between the prescriber and pharmacy/pharmacist; and
transmit auditable information to the remote terminal based on the results of the determinations.
